(19) United States
(12) Patent Application Publication
(10) Pub. No.: US 2026/0041452 A1
(43) Pub. Date: Feb. 12, 2026

(54) SYSTEM AND METHOD FOR A MANIPULATOR OF SURGICAL TOOLS

(71) Applicant: Medical Devices Corner, Inc., Menlo Park, CA (US)

(72) Inventors: Samuel Frishman, Menlo Park, CA (US); John Peter Whitney

(21) Appl. No.: 19/122,284

(22) PCT Filed: Oct. 18, 2023
(86) PCT No.: PCT/US2023/077230

§ 371 Date: Apr. 18, 2025

Related U.S. Application Data
(60) Provisional application No. 63/417,104, filed on Oct. 18, 2022.

(51) Int. Cl. A61B 17/34; A61B 17/00; A61B 34/00; A61B 90/50

(52) U.S. Cl. CPC A61B 17/3403; A61B 34/71; A61B 90/50; A61B 2017/00539; A61B 2017/00911; A61B 2017/3409; A61B 2090/506

(57) ABSTRACT

Systems and methods for an actuating needle manipulator with multiple degrees of freedom that can include a translational mechanism; a remote center of motion (RCM) mechanism coupled to a distal end of the translational mechanism; a needle insertion mechanism coupled to the distal end of the RCM mechanism; a set of actuation couplers that are coupled to the translational mechanism and the RCM mechanism for integration with at least four degrees of freedom; and an insertion actuation coupler coupled to the needle insertion mechanism.

### CROSS-REFERENCE TO RELATED APPLICATIONS

This Application is a US National Stage Application under 35 U.S.C. 371 which claims the benefit of PCT Patent Application No. PCT/US2023/077230, filed on 18 Oct. 2023, which claims the benefit of U.S. Provisional Application No. 63/417,104, filed on 18 Oct. 2022, both of which are incorporated in their entireties by this reference.

### TECHNICAL FIELD

This invention relates generally to the field of teleoperated medical devices and more specifically to a new and useful system and method for a hydraulic transmission system of teleoperation device with haptic feedback.

### BACKGROUND OF THE INVENTION

Articulating robotic arms have many applications. Increasingly, use of robotic arms and manipulators are being used and explored for medical applications. In some cases, articulating arms using integrated active components (motors, electronic control systems, sensors, etc.) are commonly used as a controllable manipulator. Other manipulators with integrated active components may be used as input devices, where manipulation of the device can be detected and used as an input. Many of these types of arms can have several varying disadvantages.

Serial mechanisms that have actuators chained together in series can have the disadvantage of the actuating elements impact the momentum of the device. This is undesirable if the mechanism is used as input as it can make it more difficult and unnatural to move. Additionally, it can be undesirable in applications where haptic feedback is important because the inertial forces of heavy actuating elements can inhibit haptic transparency and bandwidth of the overall device. It can also be undesirable to be manipulating an object with such momentum in close proximity to a patient in a medical application.

Additionally, the inclusion of active components can make such devices unsuitable for situations where metals or electronics are not permitted. For example, robotic arms with active components are not usable alongside an active magnetic resonance imaging (MRI) device and other devices.

Thus, there is a need in the teleoperated medical device field to create a new and useful system and method for a manipulator of surgical tools. This invention provides such a new and useful system and method.

### BRIEF DESCRIPTION OF DRAWINGS

FIG. 1 is a schematic representation of components of a system.

FIG. 2 is a schematic representation of component variations of a system.

FIG. 3 is a schematic representation of a system variation with a 5-bar and 6-bar multi-bar linkages.

FIGS. 4A and 4B are side and top schematic representations of a system variation with a 6-bar multi-bar linkage.

FIG. 5 is a schematic representation of a multi-bar linkage of a transitional mechanism with two connections to actuation couplers of an actuation system.

FIG. 6 is a schematic representation of integration of an actuation coupler with the translational mechanism.

FIG. 7 is a detailed schematic representation of a remote center of motion mechanism.

FIG. 8 is a schematic representation of an actuation cable system routing from a base position.

FIG. 9 is a schematic representation of an actuation cable system routing from a position distal to the translational mechanism.

FIG. 10 is a schematic representation of an actuation cable system routing from to an injection actuation coupler that is integrated as part of the end effector.

FIGS. 11 and 12 are schematic representations of actuation couplers integrated at distal positions.

FIG. 13 is a schematic representation of actuation cable system and cable routing through a rotational mechanism.

FIGS. 14A-14D are diagram representations of cable routing through the rotational mechanism in different positions.

FIG. 15 is a schematic representation illustrating a translational mechanism with a set of pulleys aligned with joints of a multi-bar linkage.

FIG. 16 is a schematic representation of a top view highlighting exemplary cable routing through a translational mechanism.

FIGS. 17-19 are flowchart representations of method variations.

FIG. 20 is a schematic representation of one generalized system variation.

FIG. 21 is a schematic representation of a system variation with a single segment of hydraulic lines extending to an output manipulator.

FIG. 22 is a schematic representation of a system variation with two segments of hydraulic lines and an intermediary active transmission interface.

FIG. 23 is a schematic representation of an exemplary system variation showing more than one input manipulator systems.

FIG. 24 is a detailed schematic representation of a system variation.

FIG. 25 is a detailed schematic representation of transmission interfaces used for one channel of a hydraulic transmission system variation.

FIG. 26 is a detailed schematic representation of a system variation with two segments of hydraulic lines.

FIGS. 27A-27C are detailed schematic representations of transmission interface variations used for one channel of a hydraulic transmission system variation.

FIGS. 28A and 28B are detailed representations of a channel of interconnected exemplary transmission interfaces using hydraulic-to-mechanical transmission mechanisms.

FIGS. 29A and 29B are schematic diagrams of exemplary implementation of the system.

FIG. 30 is a detailed schematic representation of a system variation with a digital control input and control system.

FIG. 31 is a detailed schematic representation of a system variation with a data input and control system.

FIG. 32 is a schematic diagram of an exemplary implementation showing hydraulic subsystems.

FIGS. 33A and 33B are schematic representations of system variations used across two regions.

FIG. 34 is a schematic representation of control system independently controlling subsets of channels of a hydraulic transmission system.

FIGS. 35-37 are flowchart representations of method variations.

FIG. 38 is an exemplary system architecture that may be used in implementing the system and/or method.

### DETAILED DESCRIPTION OF THE EMBODIMENTS

The following description of the embodiments of the invention is not intended to limit the invention to these embodiments but rather to enable a person skilled in the art to make and use this invention.

### 1. Overview

Systems and methods for a manipulator of surgical tools functions to enable enhanced input control and/or actuation of a surgical tool. The systems and methods enable a manipulator system that preferably includes a multi-dimensional actuating mechanism that can include a translational mechanism connected in series to a rotational mechanism. The system can further include an end effector at the distal end. The end effector may additionally be manipulated and actuated with one or more degrees of freedom.

In particular, the systems and methods may be configured and used as a surgical tool with a needle insertion mechanism that has an insertion degree of freedom (e.g., translation along some defined axis—an insertion axis). The needle mechanism could approximate or serve as a biopsy needle, an ablation needle, a probe, or any suitable device with an injection-like action.

The systems and methods may alternatively be adapted and used for other forms of end effectors. In some cases, those end effectors may include actuation or may additionally or alternatively include other controllable actions like actuation of an energy ablation tool (e.g., where activation of the ablation tool could be controlled through a digital control input, but positioning could be through the system and/or method), a cutting surgical tool (e.g., a surgical scalpel, drill or saw), grasping or retrieval tool (e.g., where grasping action could be a digital or actuating action), a suturing tool, and/or other types of tools. Herein, the examples are primarily described in the context of a needle insertion mechanism, but any alternative end effector may alternatively be used.

The systems and methods may be used in enabling a mechanism that may be used as an input and/or an output.

In one variation, the systems and methods may be configured as an input mechanism. In such an input variation, the systems and methods may be used for human manipulation, wherein the system is used as an input manipulator system. An operator (e.g., a doctor or medical practitioner) may supply input in the form of physical actuation of different degrees of freedom. This actuation may be translated via a directly coupled mechanism for translation to some output. In some variations, the input may be translated to some mechanism whereby the actuation may directly manipulate or actuate some connected device (e.g., a complimentary system used as an output). In one variation, discussed herein, input may be translated through a modified hydraulic transmission system that augments actuations and/or senses input. In some alternative variations, the input may be converted to a digital signal and/or used as an input in any suitable manner.

In particular, the systems and methods may be particularly adaptable as a haptic input device whereby haptic feedback can be provided while supplying input. The systems and methods in some variations may be coupled to a hydraulic system used (at least in part) to act on an output manipulator. Forces and feedback experienced at the output manipulator can be supplied through the hydraulic system and experienced at the input variation of the system and method.

In other variations, the systems and methods may be configured as an output mechanism. In such an output variation, the systems and methods may be used as a controllable actuator with various degrees of freedom. The systems and methods may be coupled to various sources of actuation input, which may be controlled so as to actuate the systems and methods. In some variations, the systems and methods may be coupled to hydraulic lines through which some supplied actuation input can act on the degrees of freedom. The mechanism may additionally or alternatively be controlled through some digital or electronic control system using some form of actuators to act on the mechanism (e.g., motors and/or linear actuators). In some variations, the systems and methods may act at times like an input and/or an output.

The systems and methods enable device movement with multiple degrees of freedom. In one variation, a manipulator of the systems and methods may have two translational degrees of freedom and two angular degrees of freedom with an additional translational degree of freedom for an end effector. Variations of the systems and methods may be configured to have fewer degrees of freedom. Similarly, the systems and methods may be configured with more degrees of freedom depending on the application. For example, for some types of end effectors, there may be additional degrees of freedom which could have controllable actuation.

In some variations, translation and angle adjustment may be selectively locked to isolate which degrees of freedom are variable at a given time. This may be particularly useful within a surgical tool where a practitioner may want to fix different aspects of motion at different stages. The systems and methods may integrate locking mechanisms to lock distinct degrees of freedom.

The systems and methods may additionally be configured as a substantially parallel mechanism and/or a parallel-serial mechanism. A parallel mechanism can be a mechanism characterized as one where actuators (or more generally actuator couplers or interfaces) can be grounded or otherwise positioned at a proximal side of the device (e.g., at a base of the device). For example, a series of motors or hydraulic couplers used to drive the system may be grounded or situated before the translational mechanism.

This parallel configuration of the system can be used to reduce mass of the actuating portion of the system. From variations used as haptic inputs, this means an operator does not have to move a lot of extra mass when manipulating the device. For variations used as a manipulator output, when the device is articulated, the device does not have to account for moving extra mass of the actuators on subsequent segments (such as the mass of multiple motors used in serial articulating arms). In some variations, a subset of actuators or actuation couplers for actuating the insertion degree of freedom or another degree of freedom may be mounted on a distal end of a translational mechanism and a proximal end of an RCM mechanism, establishing a parallel-serial mechanism.

The systems and methods employ a design that can enable some variations to have flexibility in the materials used within the overall device. In particular, variations of the systems and methods may not require use of active components within the physical structure. For example, the systems and methods may have any motors and electronics used to drive or sense actuation connected on the proximal end of the manipulator. The materials used within the device may be customized to the use-case. For example, the systems and methods may use scanner compatible materials which could include plastic or non-metal materials as well as non-magnetic materials, non-conductive materials, radio frequency (RF) transparent materials, non-ferromagnetic materials, and/or other material types that would be suitable for use in a restrictive environment. This may make the systems and methods compatible for use alongside MRI (Magnetic Resonance Imaging) devices, CT (Computed Tomography) devices, and/or other sensitive devices. Connected components (like motors or electronic sensors) made of materials not compatible for a particular use may be integrated the manipulator system and positioned out of range, shielded, or outside of any restricted region. As such, the device may be used for operating on a patient while the patient is being imaged in an MRI machine.

The manipulator system of the systems and methods may, in some variations, be used with a hydraulic system that is integrated with the manipulator system for transmission of actuation. In some variations, the manipulator system may be used with an enhanced hydraulic transmission system further described herein, which may provide additional actuation augmentation and/or sensing. In some variations, one or more instances of the manipulator may be connected through a hydraulic system. Actuation can be conveyed from an input manipulator system to an output manipulator system. This may or may not include an enhanced transmission system.

The system and method of the manipulator system may alternatively be used independently or in a variety of other applications such as with non-hydraulic actuators and/or in non-medical applications.

The system and method may provide a number of potential benefits. The system and method are not limited to always providing such benefits and are presented only as exemplary representations for how the system and method may be put to use. The list of benefits is not intended to be exhaustive and other benefits may additionally or alternatively exist.

As one potential benefit, the systems and methods may enable a manipulator device with multiple degrees of freedom.

As a related potential benefit, the systems and methods may additionally enable multiple degrees of freedom with a wide range of motion. For medical applications, the range of motion can be configured to enable free motion within a typical spatial region used during a procedure.

As another potential benefit, the systems and methods may be configured as a parallel mechanism, which enables grounding of all or many systems used to drive and/or detect actuation. As a result, the actuated components may be made lighter.

As another potential benefit, the systems and methods may enable more natural actuation. This feeling of the end effector being more easily manipulated may result, in part, from a potential reduction of mass by grounding actuation systems used to drive and/or detect actuation. This feeling of the end effector being more easily manipulated may also result, in part, from the range of motion of multiple degrees of freedom.

As one potential benefit, the manipulator system of the systems and methods may be compatible with a hydraulic-based actuation system. Hydraulics may be used in part to transmit actuation control to and/or from the manipulator system.

As another potential benefit, the system and method may be well suited for enhanced haptic feedback. In particular, the manipulator system of the system and method may be integrated into a system that includes similar manipulator systems for input and output manipulators. The input and output manipulators can be connected via a hydraulic-based system. In this variation, actuation input (e.g., force or pressure) can be conveyed between the two manipulator systems and any experienced resistance of feedback on the output manipulator system can be transmitted through the hydraulic-based system to the input manipulator.

### 2. Manipulator Systems

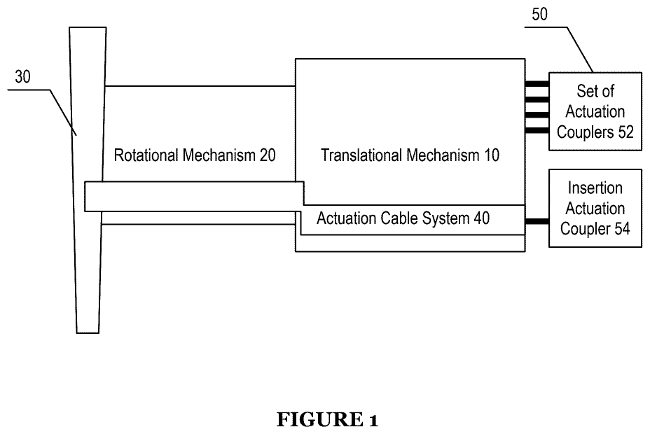

As shown in FIG. 1, a system for a surgical manipulator system (or a manipulator system for any suitable application) may include a translational mechanism 10; a rotational mechanism 20 coupled to the distal end of the translational mechanism; and optionally an end effector 30 with one or more degrees of freedom (DoF) mounted at the distal end of the rotational mechanism. The translational mechanism 10 and rotational mechanism 20 can function cooperatively to establish multiple degrees of freedom (e.g., 4 DoF) that can be individually controlled and optionally locked or controlled. For example, as an input device, a user may easily use two control points to control translation and rotation independently. The system may additionally include an actuation cable system 40 connected to the end effector 30. The actuation cable system 40 may be routed through the system to couple to the end effector 30, whereby actuation can be conveyed through the actuation cable system 40 to a degree of freedom of the end effector 30. The system may include an actuation system 50 (e.g., the actuators, connections to actuators, and/or systems to monitor actuation), which, in some variations, may be positioned proximally to the translational mechanism.

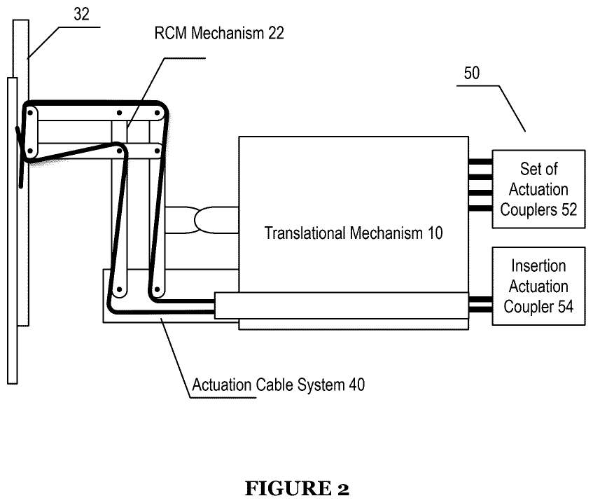

The system preferably enables actuation couplers to be integrated with the manipulator system so as to control the degrees of freedom through coupled actuators (e.g., mechanical and/or hydraulic) In some variations, the system provides remote center of motion (RCM) movements for surgical procedures. While the system may be adapted to any suitable type of surgical tool, one variation of the end effector is a needle insertion mechanism. As shown in FIG. 2, the system may include a translational mechanism 10; a remote center of motion (RCM) mechanism 22 coupled to a distal end of the translational mechanism 10; a needle insertion mechanism 32 coupled to the distal end of the RCM mechanism 20; an actuation system 50 which includes a set of actuation couplers 52 that are coupled to the translational mechanism 10 and the RCM mechanism 22 for integration with at least four degrees of freedom; and an insertion actuation coupler 54 coupled to the needle insertion mechanism 32.

In some variations, the system may be configured to enable independent degrees of freedom. That is to say that the configuration of the system with the translational mechanism and the RCM may enable translation, rotation, and needle insertion to be at least partially controlled independently. Accordingly, in some variations, the system may include a translational mechanism 10; a remote center of motion (RCM) mechanism 22 coupled to a distal end of the translational mechanism 10; a needle insertion mechanism 32 coupled to the distal end of the RCM mechanism 20; wherein the translational mechanism, the RCM mechanism, and the needle insertion mechanism include degrees of freedom that are independently lockable.

In some implementations, the independent locking of the DoF may more specifically be implemented as sequentially lockable DoF or restricted DoF. For example, translation may be set and then locked/fixed, then rotation can be set/fixed, and then insertion may be independently controlled. In a similar way, this independent control of the degrees of freedom may provide enhanced usability. For example, the system may include a first handling grip connected to the RCM mechanism 22 and a second handling grip connected to the translational mechanism 10. Moving the first and second handling grip in a coordinated fashion (translating in equivalent motions) can primarily adjust the translational position. Holding one handling grip stationary while moving the other one can result in angular actuation. Optionally, when the translational position is set, the translation may be locked (e.g., one of two stacked multi bar mechanisms locked).

In some variation, the system may additionally or alternatively use an actuation cable system 40 that is routed through or in connection with the RCM mechanism 22 and/or translational mechanism 10. Accordingly, in some variations, the system may include the system may include a translational mechanism 10; a remote center of motion (RCM) mechanism 22 coupled to a distal end of the translational mechanism 10; a needle insertion mechanism 32 coupled to the distal end of the RCM mechanism 20; and an actuation cable system 40 routed through the RCM mechanism and coupled to a degree of freedom of the needle insertion mechanism 32.

Herein, the system is primarily described as serial connections of the translational mechanism and the rotational mechanism. In some variations, an alternative multi DoF actuation mechanism may be used, which may be a single mechanism with multiple DoF or serial connection of different mechanisms. In some variations, this could be an alternative serial connection of actuation mechanisms that contribute to different degrees of freedom. For example, an alternative variation could have a rotational mechanism as the initial proximal mechanism and then a translational mechanism coupled to the distal end of the rotational mechanism.

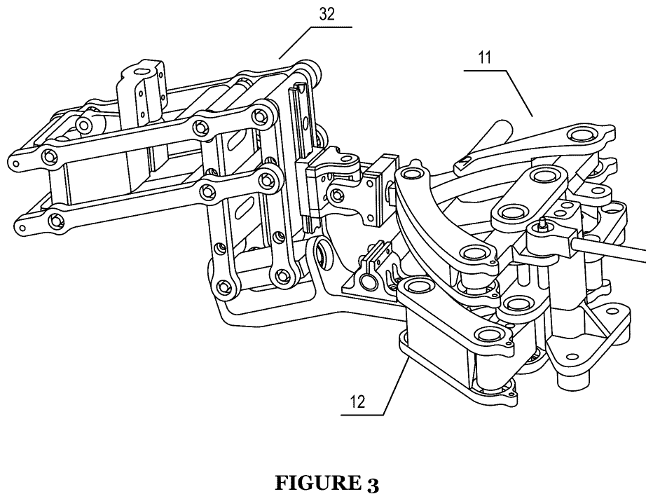

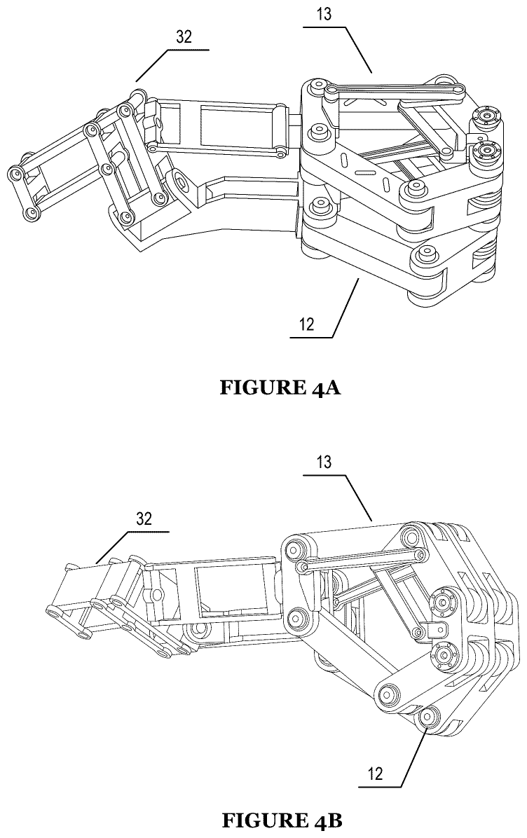

In some variations, the system includes an RCM connector to couple the RCM mechanism 22 to the distal end of the translational mechanism 10. Additionally, the translational mechanism 10 may include two multi-bar linkages arranged in a stacked parallel configuration. In other words, the translational mechanism 10 may be a mechanism with translational actuation aligned along a defined horizontal plane, and that includes a top multi-bar linkage and a bottom multi-bar linkage. For example, the system may include a 5-bar linkage 11 and a 6-bar linkage 12 arranged in a stacked arrangement, one on top of another as shown in FIG. 3. In some variations, the system may include two 6-bar linkages 12/13 as shown in FIG. 4A and FIG. 4B. In such variations, the translational mechanism can actuate or otherwise provide in part four degrees of freedom with two translational actuation and two angular degrees of freedom. The needle insertion mechanism can be an insertion degree of freedom. Herein the RCM mechanism 22 and the needle insertion mechanism 32 are used as examples of the rotational mechanism 20 and the end effector. However, these mechanisms may be interchanged with other suitable rotational mechanisms and/or end effectors 30 using design variations described herein.

As one potential feature, the system may enable grounded actuation systems. Accordingly, the system may include a set of actuation couplers 52 that are each coupled to a bar of the two of multi-bar linkages, and an insertion actuation coupler 54 coupled to the needle insertion mechanism through the actuation cable system 40. Four bar linkage or other suitable mechanical connection may be made to an input arm of a multi-bar linkage from an actuation coupler, which can be mounted or grounded on a proximal end of the main actuation mechanisms of the system.

The actuation couplers may be used for transmitting actuation input to the manipulator system through the actuation couplers, transmitting actuation input from the manipulator system to some connected system, and/or sensing actuation.

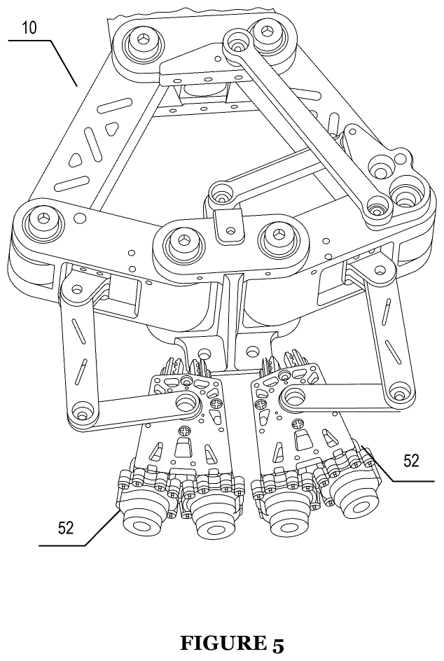

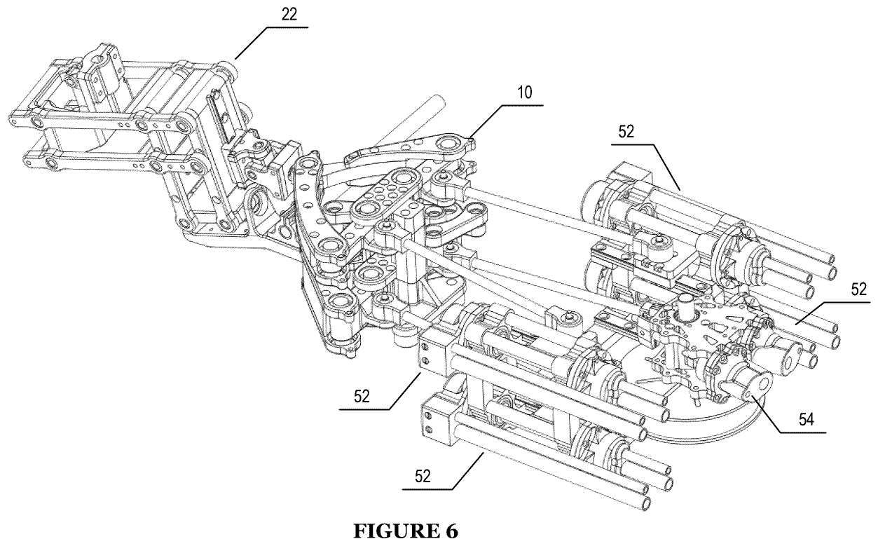

In some variations, the manipulator system may be incorporated as part of hydraulic actuation system. In one variation, the system may include a set of hydraulic lines that connect to a set of actuation couplers 52 and/or the insertion actuation coupler 54. In particular, the system may additionally include a hydraulic transmission system that includes a set of actuation transmission interfaces (functioning as actuation couplers) that are coupled to distinct bars of the two multi-bar linkages, and an insertion actuation coupler 54 that is a transmission interface coupled to the needle insertion mechanism 32 through the actuation cable system 40. As shown in FIG. 5, an actuation coupler 52 that is hydraulic-to-mechanical actuation transmission interface may connect to two different input arms of a multi-bar linkage. As shown in FIG. 6, a linear actuating hydraulic actuation coupler may be used as another variation. The connection may be made using a 4-bar linkage or any suitable multi-bar linkage, a connecting rod, using a cable or belt driven system, having an actuator directly mounted to an axis of the translational mechanism 10, and/or using any suitable coupling. Each degree of freedom may have a connected actuation coupler.

The actuation system may be coupled to the hydraulic transmission system via a hydraulic transmission interface or some other interface, whereby hydraulics are used to convey actuation to and/or from the manipulator system. In other variations, the transmission interfaces may be active transmission interfaces that are coupled to a powered actuation system. In one such variation, a system may include an input manipulator system 110 and an output manipulator system 120; and a hydraulic transmission system 130 coupling the input manipulator system 110 and the output manipulator system 120.

In this variation each of the input manipular system 110 and the output manipular system 120 may include: a translational mechanism 10; a remote center of motion (RCM) mechanism 22 coupled to a distal end of the translational mechanism 10; a needle insertion mechanism 30 coupled to the distal end of the RCM mechanism 20; and an actuation cable system 40 routed through the RCM mechanism and coupled to a degree of freedom of the needle insertion mechanism 30. Alternatively, the input manipulator system and the output manipulator system may include any variation described herein. They may also be different variations of the manipulator system described herein.

The hydraulic transmission system 130 preferably couples actuation of the input manipulator system 110 and the output manipulator system 120 at least partially through a set of hydraulic lines. The hydraulic transmission system 130 may include hydraulic transmission interfaces on either side. Each hydraulic transmission interface can couple to a manipulator system (110 or 120) through a mechanical coupling. In one variation, a set of hydraulic lines connect the two hydraulic transmission interfaces. In a direct connection variation, actuation or force applied through a degree of freedom of one manipulator system (input or output) translates from mechanical actuation to hydraulic actuation through the transmission interfaces, then is transferred through the set of hydraulic lines, and then transformed back to mechanical actuation used to act on the other manipulator system.

In some variations, the hydraulic transmission system can be modified to include capabilities to augment and/or sense actuation transferred through it. The hydraulic transmission system 130 may additionally include an intermediary active transmission interface 131 serially integrated with at least one hydraulic line. The active transmission interface 131 can enable augmentation of actuation and/or sensing of actuation.

In some variations, the manipulator system may be configured for use within a restricted environment. For example, the system may be configured for use with an active MRI device. Accordingly, the system may be made of material consisting exclusively of MRI compatible material. In other words, the entirety of the manipulator system may be fully made of MRI compatible material. The manipulator system may be similarly made of material for other situations where there may be material limitations or preference. The manipulator system may be made of plastic or non-metal materials as well as non-magnetic materials, non-conductive materials, radio frequency (RF) transparent materials, non-ferromagnetic materials, and/or other material types that would be suitable for use in a restrictive materials environment. When not used in such restrictive situations, the manipulator system may alternatively be made of any suitable material.

The translational mechanism 10 functions to provide at least one translational degree of freedom. In many variations, the translational mechanism 10 provides two translational degrees of freedom along a defined XY plane. The translational mechanism 10 may in some variations also provide 3 translational degrees of freedom. In a variation with a need injection mechanism, the injection degree of freedom may function as an alternative avenue for enabling a Z-component of translation.

The translational mechanism 10 could be any suitable type of XY plane translational mechanism like a 5-bar or 6-bar mechanism or other suitable alternative.

The translational mechanism 10 may additionally enable, in part, angular degrees of freedom, which may be conveyed to the rotational mechanism 20.

In one variation, the translational mechanism includes two multi-bar linkages (e.g., a first and second multi-bar linkages) arranged in a stacked parallel configuration. A parallel configuration can be characterized as a configuration where the linkages move in defined planes parallel to each other. As a stacked parallel configuration one multi-arm linkage is oriented in a parallel plane above the other. This may alternatively be characterized as a stacked configuration with a “top” and “bottom” multi-bar linkage aligned with a horizontal arrangement. Here top and bottom and horizontal are used as relative terms for convenience and do not limit them to being horizontally aligned relative to a gravitational or other type of reference frame.

The two multi-bar linkages 11/12 in some variations may be five or six bar linkage. In one variation, the first multi-bar linkage 11 is a five-bar linkage and the second multi-bar linkage is a six-bar linkage 12. In another variation, the first and second multi-bar linkages 11/12 are both six-bar linkages. Each of the multi-bar linkages 11/12 may be actuated in two dimensions independently.

In variations where one or both of the multi-bar linkages is a 6-bar linkage, the multi-bar linkage can include a parallelogram constraint. A grounded linkage arm and a distal linkage arm can be restrained to remain parallel because of the parallelogram constraint. When both multi-bar linkages have a parallelogram constraint, these distal linkage arms can also be maintained as parallel arms.

The parallel configuration of these two multi-bar linkages 11/12, can enable the differences in translational actuation to impact angular actuation of the system. The difference in 2D translational actuation between the multi-bar linkages 11/12 can be transferred to the rotational mechanism 20 to enable one or two degrees of angular degrees of freedom. As a whole, the translational mechanism may comprise four degrees of freedom with two translational actuation and two angular degrees of freedom.

The angular degrees of freedom may be controlled through the differential motion between the two multi-bar linkages. Differences in the XY translation of the multi-bar linkages can create angular actuation along angular degrees of freedom with axes defined perpendicularly within the plane of XY translation. These differential motions can be transformed into actuation of the rotational mechanism through couplings to each of the first and second multi-bar linkages 11/12.

For example, when the top and bottom multi-bar linkages 11/12 move together, the rotational mechanism 20 (e.g., RCM 22) may translate in the XY plane. When there is a differential motion between the top and bottom multi-bar linkages, physical couplings between the first and second multi-bar linkages result in the rotational mechanism 20 (e.g., the RCM mechanism 22) changing angle. When the rotational mechanism is an RCM mechanism 22, then the angular rotation occurs centered around an axis.

An actuation system may have action manipulate four base links at the base of the 6-bar mechanisms to control four DoFs (XY and two angles). The four base links may be connected directly or indirectly to an actuation coupler, which may be used for driving actuation, conveying actuation out to another system, and/or sensing actuation.

As shown in the detailed image of a 6-bar mechanism shown in FIG. 5, two actuators may couple to distinct arms of a 6-bar linkage. Two additional actuators preferably connect to another multi-bar mechanism used in parallel to the exemplary 6-bar linkage of FIG. 5. In particular, two 4-bar linkages may be used to couple the 6-bar mechanism to the two actuators (shown in FIG. 5 as hydraulic actuators). The actuators may be fixed as grounded elements.

The multi-bar linkages may include integrated pulleys, joints or other cable routing mechanisms or features that are used to facilitate routing of cable/tubing components of the actuation cable system 40.

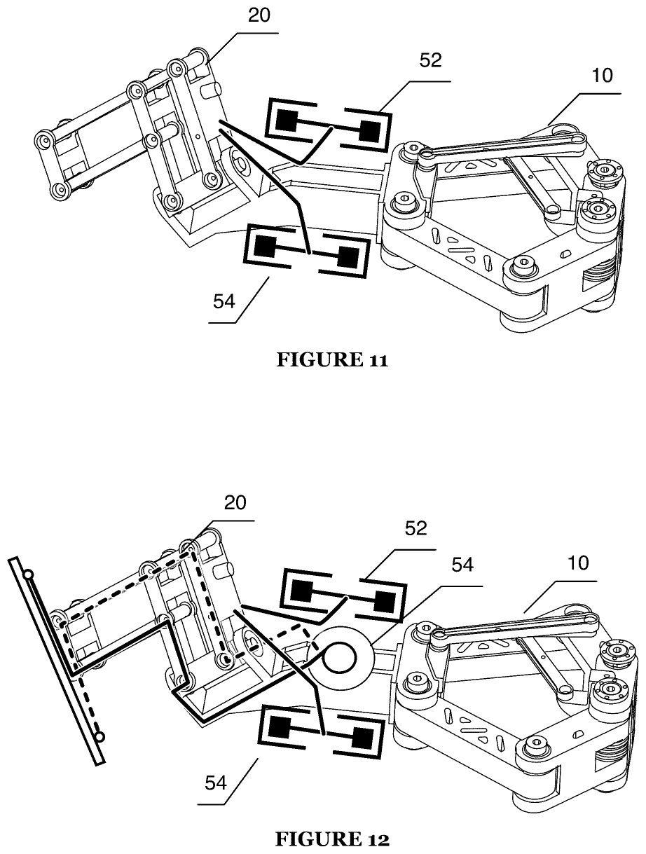

The rotational mechanism 20 functions as an angling mechanism that can facilitate rotation of the system. As discussed herein, in some cases, angular actuation may be conveyed in part, by the translational mechanism 10. However, in some variations, the angular degrees of freedom could be imparted through the rotational mechanism independently. For example, a single multi-bar linkage may be used to create two degrees of translational degrees of freedom, and the rotational mechanism may have one or more angular degrees of freedom that are coupled to the actuation system 50 for control as shown in the exemplary FIGS. 11 and 12.

In one preferred variation, the rotational mechanism is an RCM mechanism 22, which functions to restrict angular actuation to occur with it centered about a defined axis. An RCM mechanism 22 may be particularly helpful for a surgical tool where an operator may typically be wanting to operate about a small point such that a surgical tool can enter a body through one point (e.g., through a trocar).

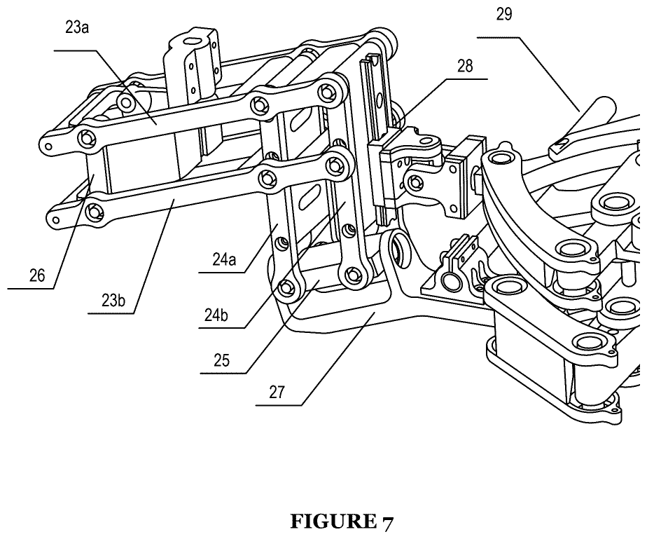

The RCM mechanism 22 can be a mechanically enforced/restrained RCM mechanism. One RCM mechanism 22 variation can be a parallelogram or rigid linkage RCM mechanism. The RCM mechanism 22 can include two interlinked parallelograms. In one variation, two pairs of interlinked parallel linkage arms 23a, 23b and 24a, 24b, can be connected to a base linkage 25 and an end effector linkage 26. The interconnection enforces parallel alignment of one pair of linkage arms 23a and 23b to the base linkage 25 and to enforce parallel alignment of the end effector linkage 26 and the other pair of linkage arms 24a and 24b as shown in FIG. 7. Multiple instances of such linkages may be used in parallel as also shown in FIG. 7. In another variation, a belt coupling between linkage arms may be used. Other suitable linkage designs may be used to establish a mechanism with RCM movement.

The RCM mechanism 22 is preferably connected to the distal end of the translational mechanism 10. The RCM mechanism 22 can be connected through a connector.

In one variation, the connector comprises a first base connector 27 connected to a distal linkage of a bottom multi-bar linkage, and a second connector 28 connected to a distal linkage of a top multi-bar linkage. As shown in FIG. 7, the first base connector 27 can have rotational degree of freedom, and the second connector 28 can linearly slide up and down and rotate with a degree of freedom. Other alternatively connectors with different couplings may be used.

The RCM mechanism 22 may have a configured downward angle relative to a plane of motion of the translational mechanism 10. The downward angle may be used orient the center of motion at a point lower than the lowest point of the mechanism.

The connector used to couple the proximal end of the RCM mechanism 22 to the distal end of the translational mechanism 10 may additionally include a handling grip 29, which functions to allow an operator to adjust the translational position of the system independent of the rotational degrees of freedom. This handling grip 29 may be particularly useful when used as an input device. For example, an operator could use the handling grip to reposition the end effector 30 in the XY plane. In another example, the operator could hold the handling grip stationary and then adjust the angle of the end effector 30. The system may additionally include an RCM handling grip or some other designed point of manipulation of the system. In some variations, the end effector 30, such as the needle insertion mechanism 32, may serve as a grip used to manipulate the RCM. As discussed above, the system may include features to have independently lockable DoF. This functions to enable isolated control of the degrees of freedom (e.g., translation, rotation, and actuation of an end effector like insertion). The lockable DoF may be implemented through exposing actuation control grips of the RCM and the translational 10 mechanism.

Moving an RCM handling grip (e.g., the needle insertion mechanism 32) and translational handling grip (handling grip 29) in a coordinated fashion (translating in equivalent translational motions) may primarily adjust the translational position. This would function to lock rotation and actuation of the end effector. Holding one control grip stationary while moving the other one can result in angular actuation. For example, holding the translational handling grip stationary, while moving the RCM handling grip can result in RCM rotational motion. This would function to lock translation and actuation of the end effector. After translation and rotation are in position, then the operator could control the end effector (e.g., the insertion actuation). The translation and angular positions would be locked/set while actuation of the end effector can be controlled independently. In such a way, the DoF have been sequentially locked through operation of the device. Such manipulation can be intuitive and natural to a user. In some variations, the system may include mechanical locking mechanism that apply brakes or other physical restrictions to lock actuation of components of the system.

The end effector 30 functions as an end device that serves as the manipulated object. The end effector 30 can be coupled to a distal end of the rotational mechanism. For example, the end effector 30 may be attached to a distal arm (e.g., an end effector arm) of an RCM mechanism 22.

In some cases, the end effector may be able to perform some action. In some such variations, the end effector can include one or more degrees of freedom of motion. In other variations, the end effector may have some controlled state, which may be controlled via an electronic and/or digital system or through some other mechanism. However, the end effector may alternatively be a passive device.

As discussed, the end effector 30 may be a needle insertion mechanism 32. However, the end effector 30 may be any suitable type of end effector such as an energy ablation tool (e.g., where activation of the ablation tool could be controlled through a digital control input, but positioning could be through the system and/or method), a cutting surgical tool (e.g., a surgical scalpel, drill or saw), grasping or retrieval tool (e.g., where grasping action could be a digital or actuating action), a suturing tool, and/or other types of tools.

The needle insertion mechanism 32 functions as a device to assist in the insertion of a needle or probe or other suitable element during a surgical procedure. The needle insertion mechanism 32 may be useful in performing biopsies and ablations, heart procedures, neurosurgical operations, and/or other operations or procedures.

The needle insertion mechanism 32 can include a translational degree of freedom used for applying an injection action (i.e., an insertion degree of freedom). This may be used so that the system can control the injection of a needle or probe into a subject. The needle insertion mechanism 32 therefore may include a moveable carriage. The carriage may be connected to the actuation cable system 40 whereby actuation along the injection axis is transferred through the actuation cable system 40. In one variation, the carriage may be mounted to the actuation cable system 40 on either end to a cable of the actuation cable system 40 such that under tension one cable can pull the carriage in one direction and another cable can pull the carriage in the opposite direction in an antagonistic manner. In another variation, a single cable may be used, and some restorative force may be used to work against the pull of the cable (e.g., a spring or gravity). In other variations, a belt may be used to drive the linear actuation. Any suitable linearly actuating mechanism may alternatively be used.

In some variations, the needle insertion mechanism 32 may additionally be rotatable about an axis. The axis of rotation of the needle insertion mechanism 32 may be defined along the axis of injection. In this way, the system may support six degrees of freedom.

For an input device variation, the needle insertion mechanism 32 may be adapted to be a main physical interaction point for a user operating the system. The needle insertion mechanism 32 may include a grip to adjust the positioning of the needle insertion mechanism 32. The needle insertion mechanism 32 may additionally include a slidable component that an operator can slide back and forth. The slidable component may be a grip or handle attached to a slidable carriage.

As an output device, the needle insertion mechanism 32 may include a fixture to hold a surgical device. The surgical device is preferably detachable such that it may be exchanged for different procedures. The surgical device may alternatively be directly integrated as part of the end effector 30. The surgical device may be adapted to be controlled for the various degrees of freedom of the end effector. For example, as an output device, the needle insertion mechanism 32 can have the needle or probe moved back and forth along a defined injection axis.

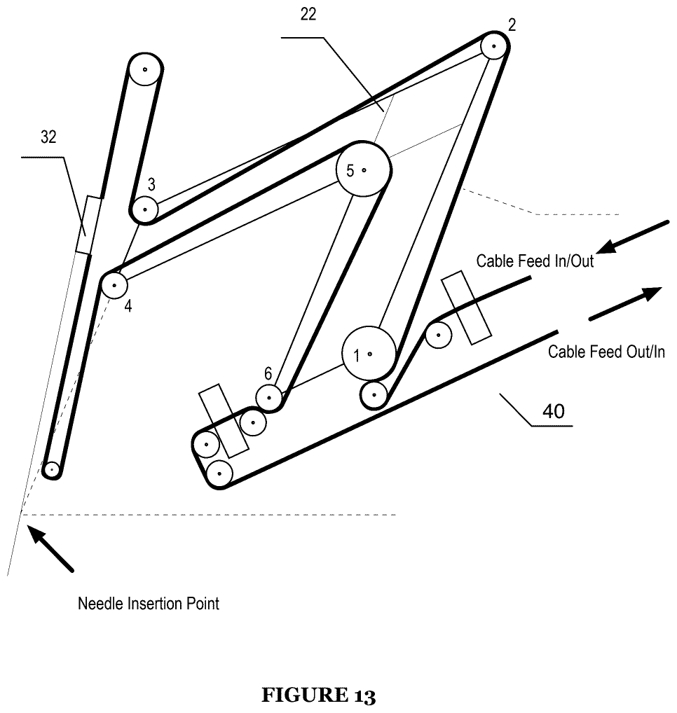

In some variations, the needle insertion mechanism 32 may be mounted with an angled offset. Accordingly, the needle insertion mechanism 32 may include an angled mounting structure establishing an angular offset from the distal end of the RCM mechanism 22, and which aligns a center point of rotation for an axis of insertion at a point below the system. The angled offset may be used to align the remote center of motion (i.e., a center point of rotation) for an axis of insertion at a point below the system and with the remote center axis of the RCM. This may make insertion point occur at a point lower than the device. This may make it easier for the system to be used on a subject. As described above, the RCM mechanism 22 may have an angled mounting relative to the translational mechanism 10 so as to direct the RCM center of motion at a point lower than the system. The angling of the needle insertion mechanism aligns the axis of insertion with the remote center of motion axis. As shown in an example of FIG. 13, the needle insertion mechanism 32 may include top and bottom pulleys of different diameters to establish an angled offset of the needle insertion mechanism 32 from the RCM mechanism and the rest of the system.

Different approaches may be used to having the needle insertion mechanism 32 being mounted with a defined insertion axis aligned with an angled offset to the RCM mechanism 22. As an alternative approach the needle insertion mechanism 32 may have the defined axis of insertion lined up in a substantially colinear with a defined axis of the remote center motion of the RCM. In one variation, this may include the needle insertion mechanism 32 connected to the RCM mechanism 22, wherein the needle insertion mechanism 32 is inset and aligned with a defined remote center axis.

The actuation cable system 40 functions as a mechanism to bridge actuation at the end effector 30 to a point towards the distal portion of the system. The actuation cable system 40 physically couples actuation of the end effector 30 with some actuation system 50. The actuation cable system 40 can additionally include cable (e.g., wire cabling or tubing) routed through the rotational mechanism 20. The cable functions as a flexible member that establishes a physical coupling between two points, and through which actuation may be transferred. Different systems may include one or more pieces of cable. In some variations, the cable can be wire cable (steel cable, synthetic cable, fiber-based cable, etc.) or tubing (hydraulic lines or pneumatic lines). The cable is not limited to these cable types and may alternatively include additional or alternative cable types such as electrically conductive wiring.

The routing of the cable preferably avoids interfering with operation of the actuation cable system 40 during actuation such as changes in cable lengths, blocking tubing from bending, excessive cable walk if wrapped around pulleys, and/or other potential issues. In some variations, the actuation cable system 40 may use wire cables under tension and/or fluid actuating system such as hydraulics/pneumatics.

The actuation cable system 40 may generally include a cable system for each degree of freedom. A cable system may include one or two cables. Two cables may be used wherein each cable works antagonistically against the other cable to apply different directions of force.

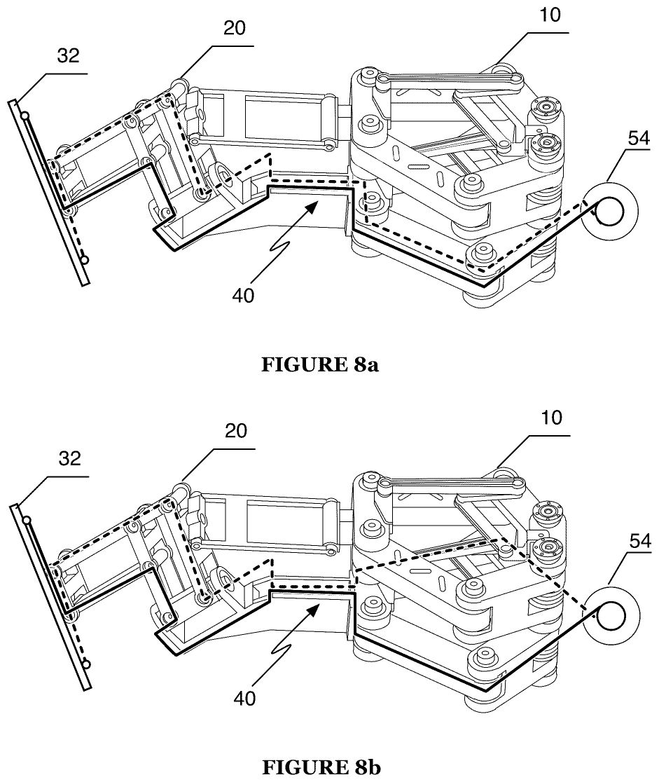

In one variation, a cable may be routed from a proximal end of the translational mechanism 10, through or around the translational mechanism 10, and through or around the rotational mechanism 30 to the end effector 30. As shown in FIGS. 8A and 8B, the cables may be routed along the same side of a multi-bar linkage or on parallel sides of a multi-bar linkage. In this variation, an interface to the actuator system 50 such as an insertion actuation coupler 54 may be mounted or integrated proximal to the translational mechanism 10. As such the injection actuation couple 54 may be grounded and may be in parallel to other grounded actuation couplers.

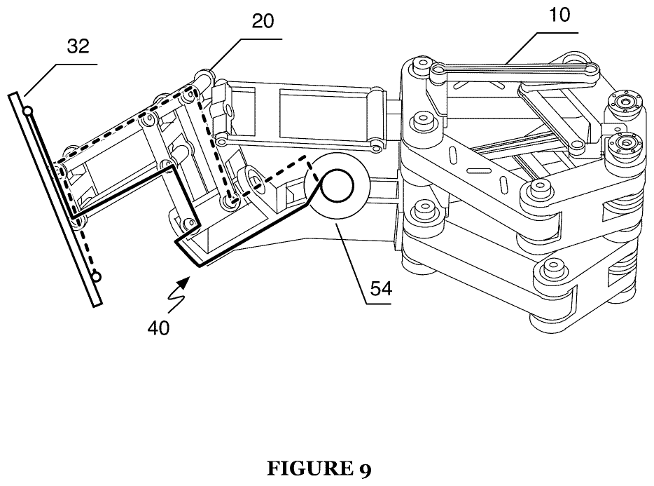

In another variation, a cable system may be routed from a proximal end of the rotational mechanism 30 and through or around the rotational mechanism 30 to the end effector 30 as shown in FIG. 9. In this variation, an interface to the actuator system 50 such as an insertion actuation coupler 54 may be mounted or integrated distal to the translational mechanism 10 and proximal to the rotational mechanism 20.

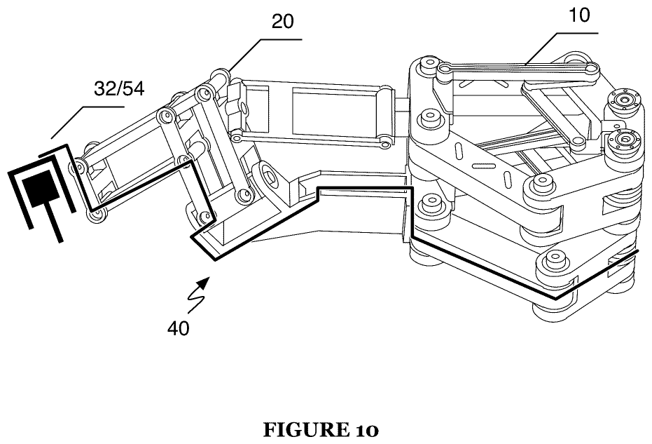

In yet another variation, the injection actuation coupler 54 may be at or part of the end effector (e.g., needle injection mechanism 32). In this variation, a suitable cable may be routed to or around the translational mechanism 10 and the rotational mechanism 20 to the end effector. As shown in the example of FIG. 10, a hydraulic line may be routed through the translational mechanism 10 and the rotational mechanism 20 to where the hydraulic action may be transformed to mechanical actuation at the injection actuation coupler 54. In a hydraulic line variation, fluid may flow through a hydraulic line along revolute joints with hydraulic rotary unions such that there is no flexible tubing. Alternatively, flexible tubing can be used to skip over the four DoF mechanism of the translational mechanism 10 and the rotational mechanism 20 and reach the distally mounted insertion actuator coupler 54 directly.

In some variations, the actuation cable system 40 may include a portion that is a wire-cable system and a portion that is a hydraulic system (or pneumatic).

The actuation cable system 30 may be a wire-driven system that uses a wire or cable coupled to an actuating component of the end effector 30. The wire cable can be under tension and use lengths of wire cable and optionally pulleys or other routing elements. In the case of a needle insertion mechanism 32, a wire cable attaches to a carriage that can slide back and forth in one dimension. As shown in FIG. 11, two cables may each be mounted at one end of an actuating element of the needle insertion mechanism 32 (e.g., a slidable carriage) along the insertion axis. A first wire cable may attach to the carriage from a first direction, and a second wire cable may attach to the carriage from an opposing direction.

In one variation, the cables may be routed around revolute joints or pulleys aligned with rotational joints of the RCM mechanism 22 as shown in FIG. 13. The routing can be configured to avoid changes in wire cable length during actuation. To avoid changes in cable length, the cables may be routed through revolute joints of the RCM in a configuration for constant cable length over set routing path. Preferably the path can be designed to be substantially constant. However, twisting of cable and manufacturing tolerances may result in some fluctuations in the length of a routing path during actuation. Constant cable length may be characterized as having less than 1-5% (in practice) of cable length change across the range of motion of the device. The theoretical change in cable length may achieve no length change or negligible length change. However, twisting (torsional twisting) of cable as well as manufacturing or design deviations from a theoretical ideal may result in some cable translation in practical implementation. The designs discussed herein can minimize or reduce such changes. Additionally, cable alignment off center of axis may induce cable length changes during twist, and may be implemented when multiple cables route along a single axis. The set routing path will generally be defined as a path from an input point of the rotational mechanism 20 to a mounting point at the end effector. For example, the set routing path can be from a point at the proximal end of the RCM to the needle insertion mechanism 32 or more specifically an actuator of the needle insertion mechanism 32 like the linear carriage. In an exemplary implementation, a first cable path routes around revolute joint 1, joint 2, and then around joint 3 before being routed to a connection point on a carriage of an insertion actuation coupler 54 as shown in FIG. 13. A second cable path routes around revolute joint 6, around joint 5, and then joint 4 before being routed to a connection point on the carriage as also shown in FIG. 13.

The revolute joints of the RCM mechanism may be configured with cooperatively compensating radii, which functions to maintain a constant length for the routing path through the RCM. Cable may be wrapped around revolute joints (which may be formed as pulleys). The cable may be wrapped around the set of revolute joints such that there is a subset of revolute joints with a first wrapping direction and a second subset of revolute joints with a second wrapping direction. This translates to some revolute joints wrapping or “eating” cable, while the other revolute joints unwrap or “feed” cable during actuation in one direction. Actuation in the other direction reverses the roles.

In one exemplary variation, the revolute joints include a set of revolute joints about which the cable is routed where two revolute joints have a first radius and a second revolute joint has a second radius, where the second radius is twice the first radius. More generally, there can be a set of revolute joints with varying radii. As the mechanism actuates, cable routed around each joint will either wrap or unwrap around the joint. The cable may be wrapped such that there is complimentary “wrapping” joints and “unwrapping” joints for any actuation. In other words, across the set of revolute joints around which the cable is routed, there are some revolute joints that will be wrapping while other revolute joints will be unwrapping. The cable wrapping across the set of joints is such that the total joint radii for wrapping joints is the same as the total joint radii for unwrapping joints. The revolute joint size design may be configured so that the portion of circumference of wrapping around a joint being wrapped is compensated by the portion of circumference that is being unwrapped.

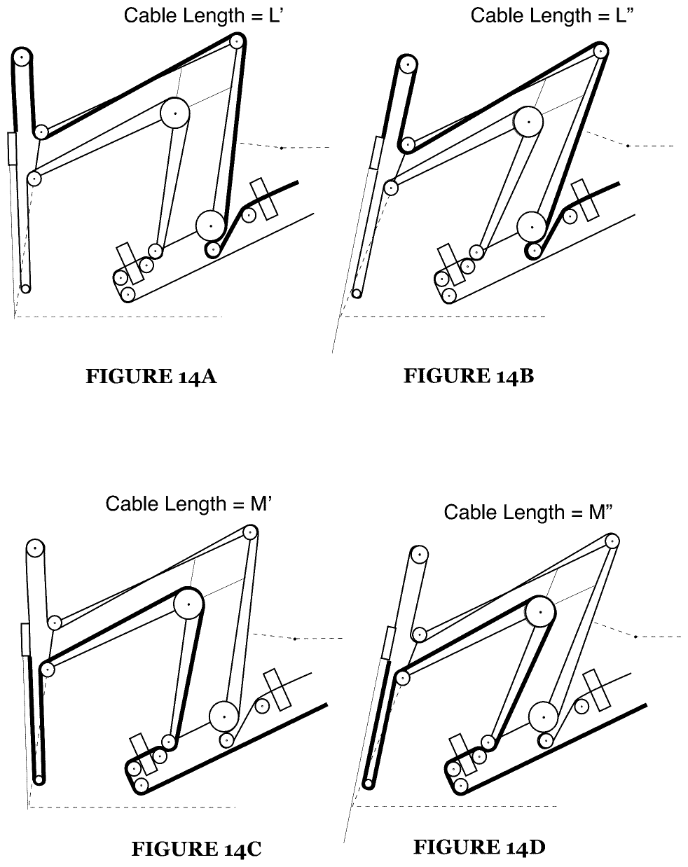

In one exemplary actuation cable system 40, a cable may be routed through revolute joints to a first joint (1) with a radius X, then around two more joints (2 and 3) each with a radius Y, where Y is half of X. Additionally as can be observed for actuation from FIG. 14A and FIG. 14B, joints 1 unwrap as joints 2 and 3 wrap. As also shown in FIGS. 14A and 14B, this length of cable is constant across a range of motion. The exemplary distance L′ will be substantially equal to L″ in FIGS. 14A and 14B. A second cable may be used as a return cable. A second cable may be routed through revolute joints to a first join (6) with a radius J, then around a second joint (5) with radius K, and then around a joint with radius J. The radius J is half of radius K. As can be observed for actuation from FIG. 14C and FIG. 14D, joints 4 and 6 unwrap as joint 5 wraps. This second length of cable is similarly kept constant across a range of motion. The exemplary distance M′ will be substantially equal to M″ in FIGS. 14C and 14D.

Other alternative cable routing options may alternatively be used. In one variation, the cable may be wrapped around a pulley at each joint. Herein, cable being “wrapped around” an object characterizes the cable substantially encircling the object, where the cable wraps around the pulley and doubles back out to the next routing point as opposed to simply routing around a joint. In this way, cable in a pulley-wrapped configuration rotates over 180° around a joint or pulley. An actuation cable system 40 with cable routed in such a pulley-wrapped configuration may similarly avoid changes in cable length during actuation.

Such cable routing designs may be particularly useful with wire cable actuation systems. However, the routing designs may be used for routing hydraulic lines as well.

The cable may additionally need to be routed through the translational mechanism. Cable routing through the translational mechanism 10 may use revolute joints with compensating radii, use pulley-wrapped configuration, and/or use other suitable solutions.

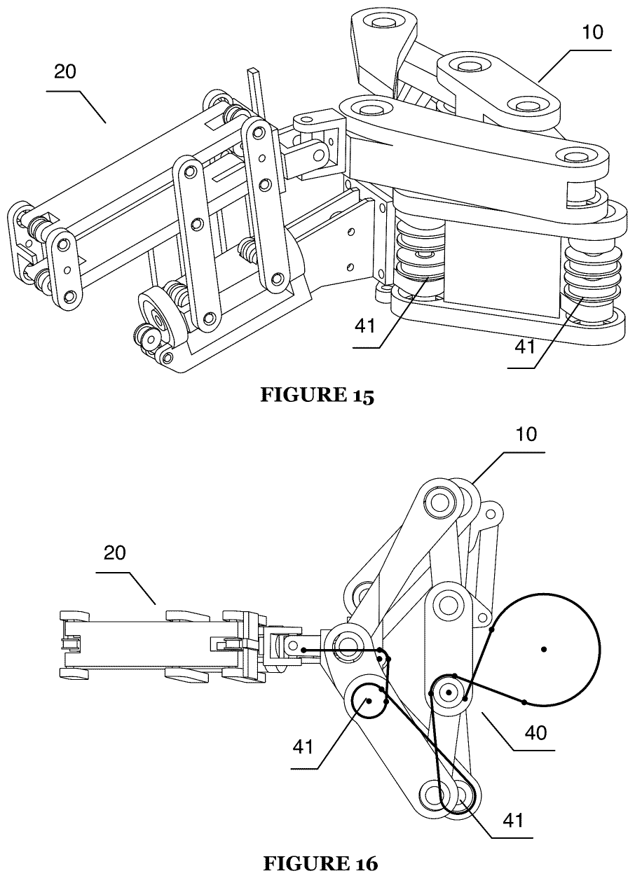

As shown in the example of FIG. 15, the translational mechanism may include a set of pulleys 41 at multiple joints of the multi-bar linkage. FIG. 15 shows two sets of pulleys to support routing of two cable lengths through the translational mechanism 10. As shown in FIG. 16, a cable may be wrapped around the set of pulleys in a pulley-wrapped configuration. The cable will preferably experience substantially no or little cable length changes. After routing through the translational mechanism 10, the cable may then route through the rotational mechanism 20.

Hydraulic tubing may be wrapped in a similar manner to wire-cabling. However, the tubing of a hydraulic system does not depend on being under tension like a wire-cable system so hydraulic lines may include slack to accommodate varying cable lengths for a direct cable routing path. Additionally hydraulic tubing preferably avoids sharp bends that can block fluid movement in the tubing. In one variation, hydraulic tubing may be integrated into the linkage arms of the translational mechanism and/or rotational mechanism 20. In one variation, hydraulic tubing may be routed through revolute joints of a mechanism via hydraulic swivels or directly with flexible tubing.

The actuation system 50 functions to transmit, convert and/or otherwise act on or in response to actuation of the system.

As an input device, the actuation system 50 may couple to actuation the system and facilitate transmitting actuation to another connected element. As an input device, the actuation system 50 may additionally or alternative sense or measure actuation converting it into some signal or data representation.

As an output device, the actuation system 50 may drive or otherwise control actuation of the manipulator system. In some variations, even when used primarily as an input device, the manipulator device may receive haptic feedback and as such the actuation system 50 may additionally be used to alter actuation or an input manipulator system.

In some variations, the actuation couplers may drive actuation using a fluid actuator such as a hydraulic system or a pneumatic system. Accordingly, the system may include a hydraulic or pneumatic actuation system that is connected to the actuation coupler. Actuation or force transferred through the hydraulic or pneumatic system can be used to drive actuation. In some variations, a transmission interface converts the fluid actuation forces into a mechanical actuation that can be transferred to some linkage or degree of freedom.

In some variations, the actuation couplers may drive actuation using a motor driven actuator. Accordingly, the system may include motorized actuators that are connected to the actuation couplers. The motorized actuators may be configured to drive actuation of the system through the connection to the actuation couplers which are connected through a physical connection to the degrees of freedom.

In some variations, the actuation couplers may sense, detect, or otherwise monitor actuation. According, the system may include sensors configured to monitor actuation through the actuation couplers. The sensors will generally be integrated into or in proximity to (e.g., within a sensing range of the sensor) the actuation couplers. The actuation couplers can experience some physical actuation proportional to actuation of a connected degree of freedom. For example, a gear or motor shaft may rotate, or a linear slider may slide back and forth.

The actuation system 50 preferably includes a plurality of actuation couplers. The actuation couplers may interface with the various degrees of freedom of the manipulator system. The actuation couplers preferably include one actuation coupler for each degree of freedom. Accordingly, the actuation couplers may include a set of actuation couplers that act on the translational and angular degrees of freedom from the translational mechanism 10 and the rotational mechanism 20. The actuation system 50 may additionally include an insertion actuation coupler as part of the set of actuation couplers or as a distinct actuation coupler. The insertion actuation coupler couples to the actuation cable system.

The actuation couplers are preferably of a similar type for each degree of freedom, but different degrees of freedom may alternatively use different actuation couplers. In one variation, the set of actuation couplers and the insertion actuation coupler are hydraulic-to-mechanical transmission interfaces that convert between mechanical actuation and hydraulic actuation. Various designs of a hydraulic transmission interface may be used as described herein. Accordingly, in some variations, a set of hydraulic lines may connect to the actuation couplers.

The placement and arrangement of the actuation couplers can have various configurations.

In one variation, the actuation system 50 includes a first set of actuation couplers 52 that controls one or more translational and angular degrees of freedom. The set of actuation couplers 52 may couple through some linkage to the translational mechanism 10.

Accordingly, in one preferred variation, the actuation system comprises a set of actuation couplers that are each coupled to a bar of the two of multi-bar linkages and an insertion actuation coupler coupled to the actuation cable system through the actuation cable system 40. Actuation may be transmitted between the set of actuation couplers and the translational mechanism 10 to affect two transitional degrees of freedom and two angular degrees of freedom. Actuation may be transmitted between the insertion actuation coupler 54 and the end effector 30 through the actuation cable system 40. Force applied to the degree of freedom of the end effector 30 can be conveyed as a force. Similarly force applied through the insertion actuation coupler 54 may actuate the end effector 30.

The set of actuation couplers, in one variation may be grounded at or before a proximal end of the translational mechanism. The insertion actuation coupler, in one variation, may similarly be grounded at or before a proximal end of the translational mechanism as shown in FIG. 8. In this variation, cable from the insertion actuation coupler 54, may be routed through or around the translational mechanism 10 to the RCM 32.

The insertion actuation coupler 54, in another variation, may alternatively be positioned at a distal end of the translational mechanism 10 as shown in FIG. 9. More specifically, the insertion actuation coupler is grounded at or after a distal end of the translational mechanism and at or before the RCM mechanism. In this variation, the system may include a connecting member that connects the insertion actuation coupler 54 to a proximal end of the translational mechanism 10. The connecting member may depend on the design of the insertion actuation coupler 54. In a variation, where the insertion actuation coupler 54 is a hydraulic transmission interface that converts between hydraulic and mechanical forces, then the connecting member may be a hydraulic line routed around or through the translational mechanism 10. In a variation where the insertion actuation coupler is an electric motorized system, then the connecting member may be an electrical line to power and/or control the electric motorized system.

In yet another variation, the injection actuation coupler may be a hydraulic transmission interface or other type of actuator positioned at or integrated with the end effector 30 as shown in FIG. 10.

In other variations, additional degrees of freedom may include actuation couplers that are moved or integrated in a region different from a base region. In an example shown in FIG. 11, two hydraulic actuators (i.e., two actuation couplers 52) for manipulating the RCM mechanism 22 are mounted distal to the 6-bar xy mechanism. Tubing routing to these hydraulic actuators can either go through revolute joints of the 6-bar linkage via hydraulic swivels, or directly with flexible tubing. The actuation couplers may connect and act on the rotational degrees of freedom of the rotational mechanism. As shown in FIG. 11, a single multi-bar linkage may be used in the translational mechanism 10 since angular rotation is actuated from the actuation couplers. As in the previous configuration, the insertion axis can be either routed fully from the mechanism base or with a hydraulic actuator mounted at the same location as these actuators for the RCM mechanism as shown in FIG. 12.

### 3. Manipulator Methods

A method for a manipulator of surgical tools can include providing and/or controlling a manipulator system such as described. The method may function to enable a manipulator system that can be used as an input and/or an output.

The method may further be used in combination with a method for a hydraulic transmission system of a teleoperation device with haptic feedback, which is also described herein. In such a variation, the method for the manipulator may be used for one or multiple manipulator systems used with a hydraulic transmission system.

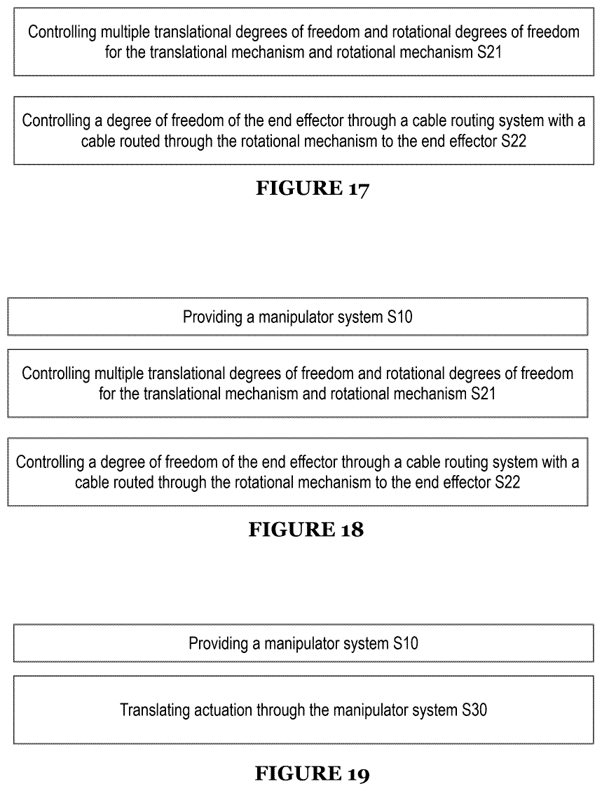

The method may include, at a manipulator system such as described herein with a translational mechanism coupled to a rotational mechanism coupled to an end effector, controlling multiple translational degrees of freedom and rotational degrees of freedom for the translational mechanism and rotational mechanism S21, and controlling a degree of freedom of the end effector through a cable routing system with a cable routed through the rotational mechanism to the end effector S22 as shown in FIG. 17.

The method may additionally include assembling, configuring, or otherwise providing the manipulator system. Accordingly, as shown in FIG. 18, the system may include providing a manipulator system S10 which comprises providing a translational mechanism with at least two translational degrees of freedom, coupling a rotational mechanism to a distal end of the translational mechanism, coupling an end effector with at least one degree of freedom to the distal end of the rotational mechanism, and connecting an actuation cable system to the degree of freedom of the end effector by routing a cable through the rotational mechanism, wherein the rotational mechanism articulates to provide two angular degrees of freedom of the end effector; and controlling the two translational degrees of freedom and two rotational degrees of freedom for the translational mechanism and rotational mechanism S21, and controlling the degree of freedom of the end effector through the cable routing system S22.

Providing the manipulator system S10 may be used to configure a manipular system with any of the variations described herein. In some variations, the rotational mechanism can be an RCM mechanism as above and the end effector may be a needle insertion mechanism. In one variation, providing the manipulator system can include adjusting angular offset of the end effector, and calibrating or setting the center point of motion (e.g., the insertion point) for the needle insertion mechanism.

Controlling the two translational degrees of freedom and two rotational degrees may include actuating and/or receiving actuation input from some actuation system. Controlling can be transferring actuation or force to the manipulator system to promote actuation. Mechanical actuation and/or hydraulic actuation may be used to control the degrees of freedom.

In other variations, a method for using the manipulator system as an input device. In such a variation, the manipulator system may include translating actuation through the manipulator system S30. As shown in FIG. 19, the method for an input manipulator system can include providing a manipulator system S10 and translating actuation through the manipulator system S30, which can include translating horizontal two translational degrees of freedom through the translational mechanism, translating two angular degrees of freedom through articulation of the rotational mechanism, and translating an end effector degree of freedom through the actuation cable system. Actuation is preferably transferred physically through distal ends of the system to a proximal side, where sensors or other mechanisms may be used to receive and/or detect the actuation and/or input to the manipulator system. In one variation, the manipular system is coupled to a hydraulic transmission system, and actuation of the manipulator system is converted from mechanical actuation to hydraulic actuation and then transmitted through the transmission system.

### 4. Hydraulic System Overview

As discussed, the manipulator system(s) and/or method(s) described herein may be used with a hydraulic transmission system. The hydraulic transmission system may alternatively be used without the manipulator system or with a different manipulator system.

Systems and methods for a hydraulic transmission system of a remote haptic interface function to use an integrated active transmission interface that can provide power-assisted actuation and/or sensing capabilities. The systems and methods use a set of hydraulic lines to translate forces and actuation between input and output manipulators. As an intermediary component between the input and output, an active transmission interface can be used to augment the hydraulic system with dynamic augmentation of actuation and/or forces transmitted within the hydraulic lines and/or with added sensing capabilities.

In some variations, the systems and methods may include a human controlled input manipulator system (e.g., a mechanical controller) that is used to control and at least partially drive a remote output manipulator system (e.g., an end effector). The systems and methods may additionally enable transmission of haptic feedback from the output to the input manipulator, which may function to provide responsive tactile haptic feedback to the operator of the input manipulator system. The systems and methods may be used to enable remote operation of some manipulator device that can be used in medical procedures or other areas of use.

The systems and methods are specially configured to enable the combination of an active electronic-based system while being configured such that material requirements for the different components enable it to be used in situations suitable for operation of a manipulator in restricted environments such as alongside medical scanners like an MRI machine. As an active electronic-based system the systems and method may benefit from active motor control, digital control of such motors, and/or digital sensing. The special configuration and use of hydraulic actuation is used to allow spatial segregation of materials based on the environment of use. This may make the systems and methods compatible for use alongside MRI (Magnetic Resonance Imaging) devices, CT (Computed Tomography) devices, and/or other sensitive devices. For example, the system's and method's approach to integrating electronic components can enable the distal end of the device (e.g., the output manipulator and the output hydraulic lines) to be made of MRI compatible materials. The non-compatible materials may be integrated within components that can be positioned out of range, shielded, or otherwise safe from the sensitive device(s). As such, the device may be used for operating on a patient while the patient is being imaged in an MRI machine.

In particular, the systems and methods use a hydraulic transmission system with an active transmission interface that couples two haptic manipulators. The active transmission interface in one variation includes a powered actuation system (e.g., an electric motor). The powered actuation system can be configured within the hydraulic transmission system such that it can intervene to actively augment the actuation output and/or haptic feedback and actuation (i.e., forces) that are translated (bidirectionally) between input and output manipulators.

A powered active transmission interface may be used to correct or adapt haptics. This may, for example, be used to increase or decrease the resistive force felt by an operator using the input manipulator system. In some cases, this may be used to counteract resistive forces resulting from the hydraulic system and/or other mechanisms used in the system to provide a more natural or realistic translation of haptic feedback from the output to the input.

The systems and methods can additionally enable dynamic changing of control modes. The configuration of the systems and methods can enable a range of control modes ranging from passive control (where powered active control of the active transmission interface is disabled) to semi-automated (e.g., using combination of operator actuation input and powered actuation) and/or fully automated (e.g., using powered actuation without actuation input from the input manipulator system).

In some variations, the systems and methods may enable the device to be manipulated fully manually (i.e., passive mode), with power assist (e.g., using powered actuation within the active transmission interface), and/or in a fully autonomous mode, all within the same device. A device's ability to have fully manual and fully automated modes within the same device may provide a number of usability and reliability benefits.

In some variations, the active transmission interface may additionally or alternatively include a sensing system that is used to monitor and sense the actuation and state of the system. A sensing system may be integrated to create a sensor-enabled active transmission interface. It may also be integrated in combination with a power-assisted variation to enable a powered and sensor-enabled active transmission interface. A sensor-enabled active transmission interface may be used to monitor the state of the device. This may also be used to collect data on the use of the device during different variations, which could be used in enabling data-driven dynamic control of the device.

Herein, the transmission system with powered and/or sensing capabilities is primarily described as an intermediary subsystem oriented between the input and the output. In some variations, a powered actuator variation or sensing variation of the transmission interface may be configured at an input side of the hydraulic transmission system. Configuration of the system to have an intermediary transmission interface away from the output may enable better actuation control and/or sensing, while keeping the output manipulator composed of materials and elements that can be used within the bore of an MRI machine.

The systems and methods may further include components that enable a highly flexible and versatile system that can be used in a variety of ways. For example, the systems and methods may be used for enabling remote operation of a manipulator with one or more degrees of freedom. When there is a plurality of degrees of freedom within a manipulator, the different degrees of freedom may be controlled individually or in groups. For example, a given subset of degrees of freedom may be locked, made to operate passively, or to have any form of semi-or fully-automated modes of control. Additionally, the systems and methods may include design features that enable reconfiguration of the system so that it can be adapted to different designs of manipulators or different uses. With such flexibility and modularity, the systems and methods may be highly adaptable for use in a medical setting. The various manipulators, hydraulic lines, and transmission systems may be configured so that they can be disconnected and reconnected without substantially loss of pressure to the hydraulic system. This may be used to quickly prepare or clean up after a procedure. For example, some components could be swapped out between procedures for sanitation reasons. The modular configuration may also enable the device to be customized for different usages. For example, the device could be reconfigured to be a fully passive system (e.g., without an active transmission interface), to include a different output manipulator (e.g., end effectors), and/or to use a different input manipulator.

The systems and methods are preferably used in enabling a device that can be used for performing medical procedures. In one particular application, the systems and methods may be used for performing procedures on a patient with live MRI monitoring of the patient. The output side of the device can be made free of metals or electronics such that it can be used on a patient within the bore of an MRI machine. The human operator and the active transmission interface can be positioned away from the MRI machine. In some cases, the input manipulator system and the active transmission interface and/or other components (that are not compatible with use near a scanning device) can be located in a shielded environment. The hydraulic lines can be the mechanism through which remote actuation control is transported. The device may additionally or alternatively be used in combination with other imaging technologies such as CT scanners, ultrasound imaging, or other imaging technologies. The systems and methods are not limited to be using in combination with such imaging technologies. The systems and methods provide numerous advantages such as enhanced haptic feedback, dynamic control features, and/or possibility for direct human mechanical control, which have advantages over other offerings even without using the device in a material-restricted application. The ability to perform procedures simultaneously with advanced imaging can enable novel user interfaces such as providing AR/VR user interfaces for use while using the system and method.

The systems and methods may be adapted for any suitable types of input control and output manipulation. In one variation, the systems and methods are used to enable performing biopsies and ablations, heart procedures, neurosurgical operations, and/or other operations or procedures.

Herein, the systems and methods are described primarily as having an input portion of the device (the portion used by one operator) and an output portion of the device (the portion performing the intended actuation). The systems and methods may alternatively be adapted for having multiple inputs (used by multiple people) and/or having multiple manipulators (multiple distinct end effectors).

Additionally, in some variations, the system may be used in a context where there is less distinction of an input and output, and instead actions/feedback are transmitted between two or more manipulators bidirectionally. Accordingly, herein, references to input and output are used to characterize general roles of different portions of the system and method for convenience. Actuation may be bidirectional such that supplied direction or force input may be supplied from either input or output and similarly affected or delivered force may be delivered to either input or output. As such input or output may alternatively be characterized with alternative labels such as first and second or controller and end effector.

The system and method may provide a number of potential benefits. The system and method are not limited to always providing such benefits and are presented only as exemplary representations for how the system and method may be put to use. The list of benefits is not intended to be exhaustive and other benefits may additionally or alternatively exist.

As one potential benefit, the systems and methods may enable teleoperation of a medical surgical/operation tool with live imaging. As discussed, the systems and methods can eliminate incompatible materials and components from the output manipulator such that the device can be used, for example, inside of the bore of an MRI machine. Physical operation on a patient may then be performed while live MRI imaging is performed. This could similarly be applied to other types of imaging. This could result in procedures being more successful and/or being performed in less invasive ways.

As another potential benefit, the systems and methods may provide a solution that offers realistic and useful haptic feedback. The hydraulic system can preferably translate haptic feedback from the output manipulator back to the input manipulator. This can be important to providing a doctor with enhanced control while performing a procedure. Furthermore, a powered active transmission interface may be used to correct for resistance or non-linearities in the hydraulic transmission such that haptic feedback is controlled. This may be used to provide more of a more realistic feeling to actions and tactile feedback. This may additionally or alternatively be used for synthetically altering the haptic feedback based on various conditions. For example, synthetic walls or barriers could be simulated by the device to prevent actuating into restricted areas.

As another potential benefit, the device of the systems and methods may be operated in various modes, ranging from a purely passive mode (primarily using the hydraulic transmission system to passively drive the output) to a fully autonomous mode, where the powered active transmission interface is used to drive the output. Power-assist modes may also be used to provide features like removing tremor from the user input, preventing or restricting potentially unintended inputs (e.g., sudden jerking motions), translating input motions to a different output motion (e.g., scaling up or down amount of movement), or for other operations. The systems and methods may additionally dynamically alter modes of operation. For example, the systems and methods may switch between different control modes, enable multiple modes simultaneously, use different modes for different degrees or freedom, and/or dynamic shifting of modes. These modes and/or the changing of modes may be manually or dynamically enabled and disabled.

As another potential benefit, the systems and methods may have actions of the device tracked using an integrated sensing system. Data can be collected as an operator uses the device. This may be used to track, monitor, and/or model actions when performing a procedure. Collected data may be used to customize the operation of a device for a user. The collected data may additionally or alternatively be used in enabling automated control of the output.

As another potential benefit, the systems and methods may be a modular and easily configured device. This configurability of the device may provide many affordances helpful when used in a medical setting. For example, the systems and methods may have swappable end effectors for fast setup and breakdown before a procedure. In some variations, the systems and methods may make some components using cost effective manufacturing techniques and materials so that the system components could be disposable or more easily washed for meeting the sanitation challenges of a medical setting.

### 5. Hydraulic Systems

A system for a hydraulic transmission system of a teleoperation device with haptic feedback may include a hydraulic transmission system that can interface with at least two manipulator systems. The hydraulic transmission system preferably connects to and transmits actuation/haptic control between an input manipulator system and the output manipulator system. In some variations, the system may include the manipulator system(s). In some variations, the system may include interfaces to engage with a compatible external manipulator system (e.g., where the system is provided without an external manipulator system). Herein, the system is described including description of the external manipulator system, but the system is not limited to always including such a component.

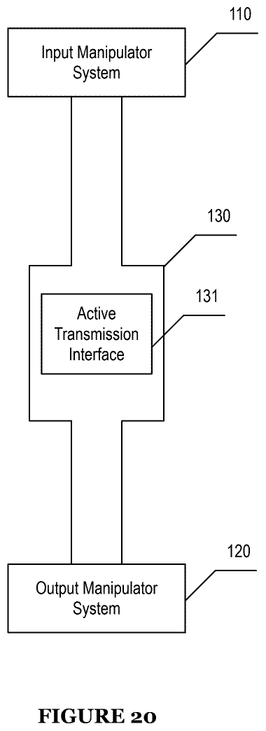

In one variation shown in FIG. 20, the system may include an input manipulator system 110, an output manipulator system 120, and a hydraulic transmission system 130. The hydraulic transmission system 130 preferably couples actuation of the input manipulator system 110 and the output manipulator system 120 at least partially through at least one hydraulic line. The hydraulic transmission system 130 preferably includes an intermediary active transmission interface 131 serially integrated with the at least one hydraulic line. The active transmission interface 131 can enable augmentation of actuation and/or sensing of actuation.

The system uses multiple “interfaces” between hydraulic systems, mechanical systems, active motor controlled and/or sensored systems, and/or digital/computer-controlled systems. These components are integrated to enable enhanced capabilities at the distal “output” manipulator that traditionally are not possible within restricted environments such as inside of a bore of an MRI machine during use. The output portion of the system is preferably used to extend into a restricted region, and the materials used on the system components in this region may be limited to compatible materials. The restricted region in one potential application can be a region where a scanner is being used. The other components may be positioned or housed in a region without the restrictions (e.g., a shielded environment). Use of electronics and other materials may be used for components in the shielded/unrestricted environment. The use of hydraulics as a mechanism for conveying actuation is used to bridge the distance between these environments, enabling segregation of components.

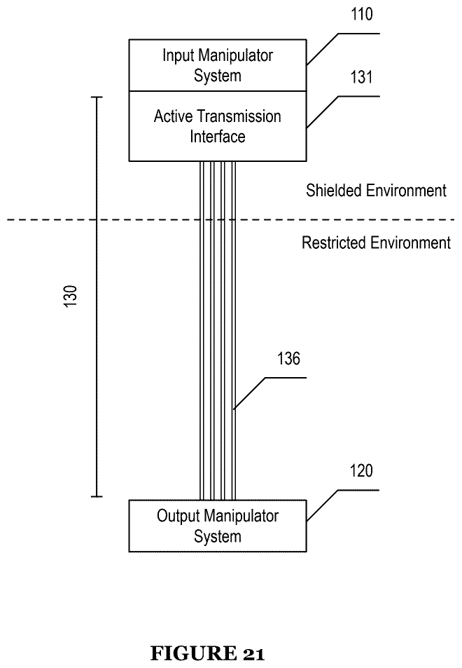

The topology or network of interconnections of the system may be configured in a variety of ways. As shown in FIG. 21, in one variation, a set of output hydraulic lines 136 are used to couple actuation output from the active transmission interface 131 to an output manipulator system 110. The active transmission interface 132 may be coupled to an input manipulator system 110 in some manner.

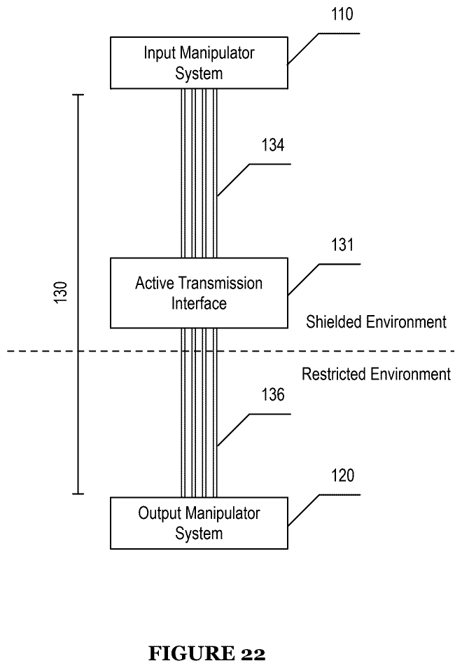

Alternative networks of interconnections may alternatively be used. As shown in FIG. 22, in another variation with two legs of hydraulic lines, a set of input hydraulic lines 134 may be used to couple actuation between the input manipulator system 110 and the active transmission interface 131, and a set of output hydraulic lines 136 may be used to couple actuation between the active transmission interface 131 and the output manipulator system 120. This variation may function to enable a symmetrical system.

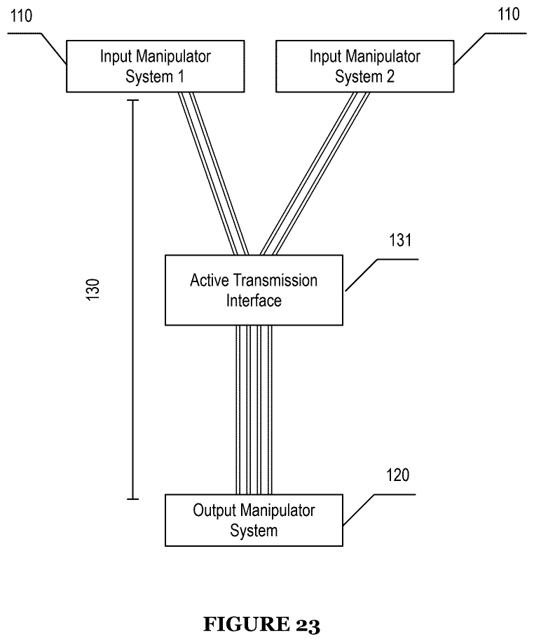

The systems are described herein primarily as including an input and an output. As discussed, the roles of these components may not strictly always be input of instructions and output as in the resulting action. Actuation and forces can be bidirectionally applied such that forces at the input are translated to the output and similarly forces at the output may be translated to the input. Additionally, the system is not limited to only having a single input and/or output. There may be multiple input manipulator systems 110 and/or multiple output manipulator systems 120. As shown in the example of FIG. 23, one variation may include two distinct input manipulator systems 110. The two input manipulator systems may be used to enable different aspects of the output to be controlled by two distinct users. Herein, the examples primarily describe a topology of a single manipulator input to a single output manipulator, but any suitable topology of inputs and outputs may be configured.

In one generalized variation, the system can include an input manipulator system 110; an output manipulator system 120; a hydraulic transmission system 130 comprising: an intermediary active transmission interface 131 serially integrated between the input manipulator system 110 and the output manipulator system 120, the active transmission interface 131 comprising a powered actuation system 132, an output transmission interface 137 that is coupled to the output manipulator system 120, and a set of output hydraulic lines 136 that connect on one end to the active transmission interface 131 and on the opposing end to the output transmission interface 137.

The system is preferably capable of having the active transmission system 131 being used to passively transfer actuation through it without intentional and active intervention/augmentation by a powered actuation system 132. Accordingly, force applied to the input manipulator system 110 or the output manipulator 120 is mechanically transferred through the active transmission interface with optional intervention (e.g., augmentation or autonomous control) by the powered actuation system. The intervention may be active augmentation or autonomous control whereby motors or other actuation systems are actively powered and driven for applying some active change to forces (e.g., actuation) transferred through the active transmission interface 131. The system may be used to transfer and augment actuation and force from input to output and/or from output to input. Accordingly, input applied to the input manipulator 110 is mechanically reflected in the output manipulator 120 via the active transmission interface 131 with or without intervention (e.g., active augmentation) by the powered actuation system 132. Similarly, input applied to the output manipulator 120 is mechanically reflected in the input manipulator 110 via the active transmission interface 131 with or without intervention (e.g., active augmentation) by the powered actuation system 132.

In addition to or as an alternative to the powered actuation system 132, the system may include a sensing system 133 coupled or integrated with the active transmission interface 131 (or the powered actuation system 132). The sensing system 133 can be configured to measure actuation transmitted through the hydraulic transmission system.

The system can additionally include a control system 140 that includes one or more computer processors and computer readable mediums (e.g., non-transitory computer readable mediums) storing instructions that when executed by the one or more computer processors cause the control system to perform various operations. These operations may be used in controlling the powered actuation system 132, receiving data from the sensing system 133, and/or interacting with other system components or external systems. The control system 140 can include configuration to control the powered actuation system 132 to at least partially augment hydraulic actuation/haptic interactions translated through the active transmission interface 130 between the input manipulator system 110 and the output manipulator system 120.

The system may be configured for one or more degrees of freedom. Each degree of freedom may be supported by having a distinct pathway coupling a mechanical degree of freedom of an input manipulator system 110 to a degree of freedom of the output manipulator system 120. Each degree of freedom accordingly can include a distinct “channel” (or pathway) made up of series of interconnected transmission interfaces (e.g., active transmission interface, output transmission interface and optionally input transmission interface) and distinct segments of hydraulic lines. Each degree of freedom may additionally include an active transmission interface instance with a distinct coupling to the powered actuation system 132 (and distinct motor coupled to an active transmission interface) and/or sensing system 133. A shared control system or individual control system may be used in controlling each degree of freedom. Herein, an individual subsystem used to manage one degree of freedom may be referred to as a channel or pathway of the hydraulic transmission system. Each channel may be configured in substantially similar way for a given system implementation. However, the channels of the hydraulic transmission system 130 may be configured with differing designs and features depending on the implementation. The channels may be controlled and augmented individually and independently.

The system in one preferred area of use is with a medical scanner like an MRI device. The system can enable teleoperation of some manipulator (e.g., an articulated syringe, arm, probe, etc.) within the medical scanner even during use. Different components of the system may be situated within an environment according to their function and design. In one variation, the input manipulator system 110 and the intermediary active transmission interface 131 are positioned within a shielded environment (e.g., an unrestricted environment), and the output manipulator system 140, and at least part of the set of output hydraulic lines 136 and the output transmission interface 137 are in a scanning region of a medical scanning device. The output manipulator system 140 in addition to the output transmission interface 137 and at least part of the output hydraulic lines 136 within a restricted region may be made of material compatible with the restricted environment (e.g., compatible with operation of an MRI machine).

Accordingly, in a system for remote operation of a device (e.g., a teleoperated device) within a medical scanner, the system may include an input manipulator system 110 situated in a shielded region (or unrestricted region) outside of the medical scanner; an output manipulator system 120 situated within a scanning region of the medical scanner; and a hydraulic transmission system comprising: an intermediary active transmission interface 131 serially integrated between the input manipulator system 110 and the output manipulator system 120 and within the shielded region, the active transmission interface 131 comprising a powered actuation system 132, an output transmission interface 137 that is coupled to the output manipulator system 120, and a set of output hydraulic lines 136 connecting or coupling to the intermediary active transmission interface 131 in the shielded region and the output transmission interface 137 in the scanning region (e.g., coupling to the output manipulator system 120).

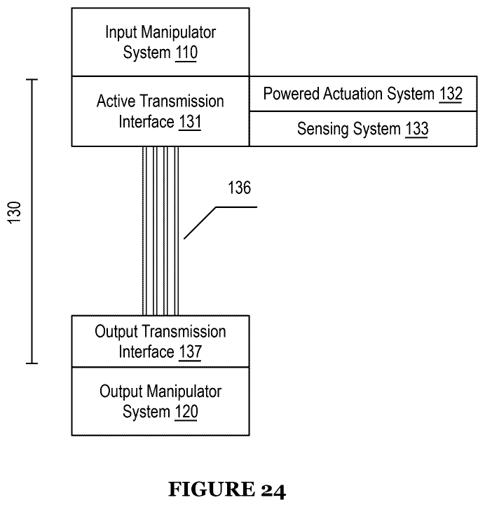

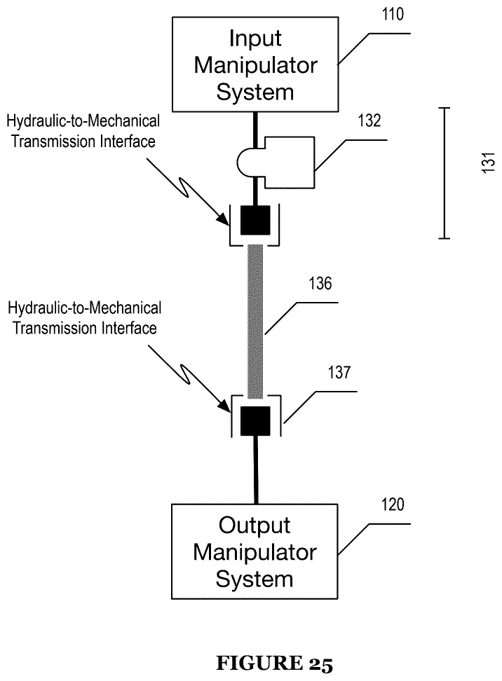

In one system variation, the system may integrate the active transmission interface 131 with a more direct mechanical coupling to the input manipulator system 110. This variation may include a single leg of hydraulic lines in the form of the output hydraulic lines 136 as shown in FIG. 24. The system can include an input manipulator system 110; an output manipulator system 120; a hydraulic transmission system 130 comprising: an intermediary active transmission interface 131 serially integrated between the input manipulator system 110 and the output manipulator system 120, the active transmission interface 131 comprising a powered actuation system 132, an output transmission interface 137 that is coupled to the output manipulator system 120, and a set of output hydraulic lines 136 that connect on one end to the active transmission interface 131 and on the opposing end to the output transmission interface 137, and wherein the input manipulator system is directly mechanically coupled to an input interface of the intermediary active transmission interface. As shown in a FIG. 25, for one channel, the active transmission interface 131 may at an input interface mechanically couple to the input manipulator system (e.g., through a mechanical linkage) and at an output interface couple to the output hydraulic lines (e.g., using a hydraulic-to-mechanical transmission interface to convert between mechanical actuation and hydraulic actuation, such that actuation can be conveyed to/from the output manipulator).

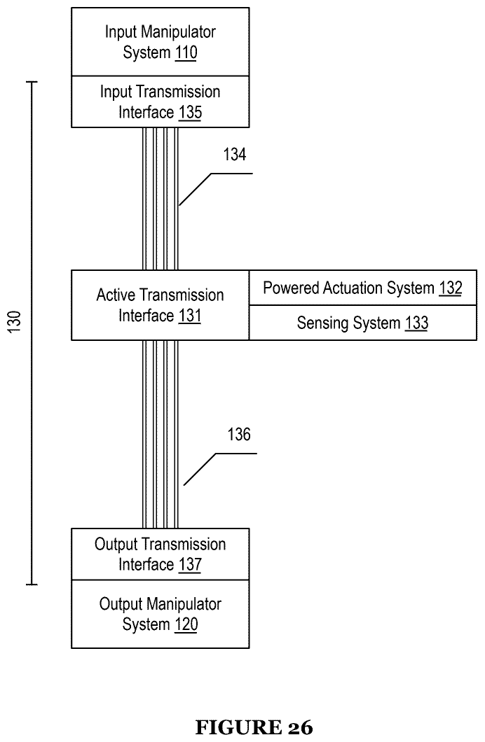

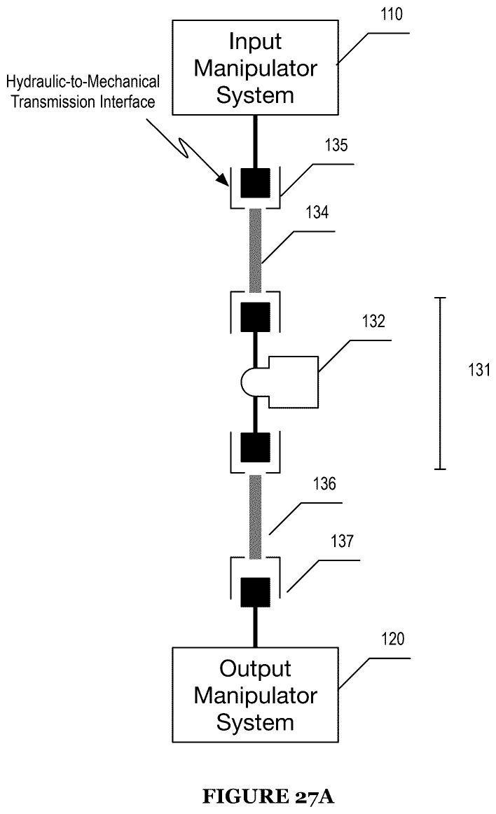

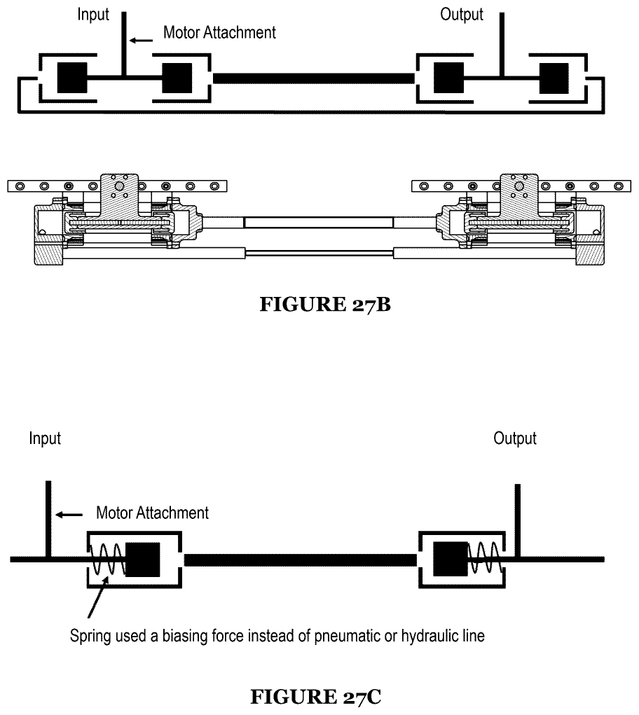

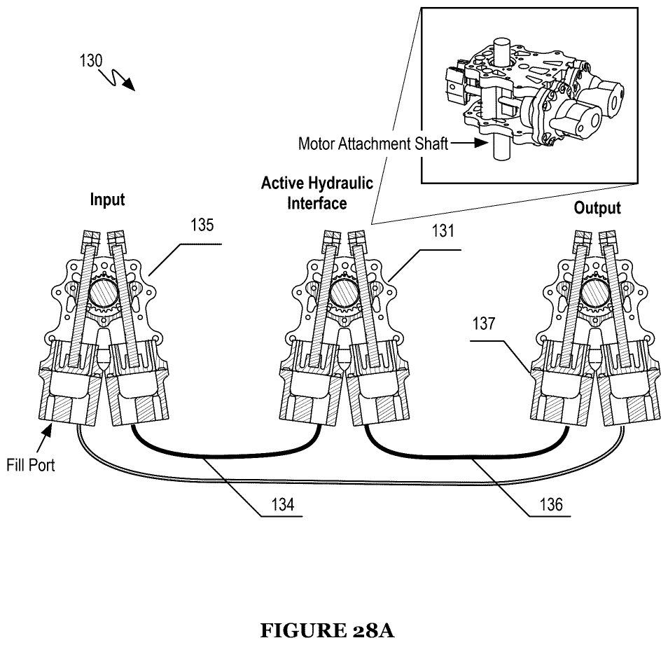

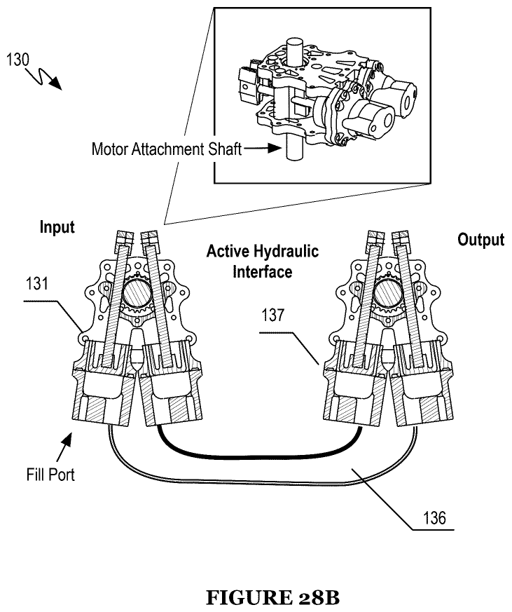

In another system variation, the active transmission interface may be an intermediary component integrated in series within hydraulic lines between the input and output. This variation may include two legs of hydraulic lines with a set of input hydraulic lines and a set of output hydraulic lines as shown in FIG. 26. The system can include an input manipulator system 110; an output manipulator system 120; a hydraulic transmission system 130 comprising: an intermediary active transmission interface 131 serially integrated between the input manipulator system 110 and the output manipulator system 120, the active transmission interface 131 comprising a powered actuation system 132, an input transmission interface 135 that is coupled to the input manipulator system 110 and a set of input hydraulic lines 134 that connect on one end to the active transmission interface 131 (e.g., via a hydraulic-to-mechanical input interface) and on the opposing end to the input transmission interface 135, and an output transmission interface 137 that is coupled to the output manipulator system 120, and a set of output hydraulic lines 136 that connect on one end to the active transmission interface 131 and on the opposing end to the output transmission interface 137. As shown in FIG. 27A, for a single channel, the active transmission interface 131 may at an input interface couple to the input hydraulic lines (e.g., using a hydraulic-to-mechanical transmission interface to convert between mechanical actuation and hydraulic actuation, such that actuation can be conveyed to/from the input manipulator) and at an output interface couple to the output hydraulic lines. As shown in FIGS. 28A and 28B, a set of hydraulic-to-mechanical transmission interfaces may be connected for one channel to facilitate translation between hydraulic actuation and mechanical (e.g., linkage-based) actuation. These transmission interfaces can expose mechanical components that can be used for integrating the powered actuation system 132 and/or the sensing system 133.

As shown in FIG. 27B, a single channel for a system with a single leg of hydraulics, may include two hydraulic lines. In some variations, a pneumatic line may be used in place of one of the hydraulic lines. These may be used to couple two hydraulic interfaces that have a hydraulic-mechanical mechanism that facilitates conversion between hydraulic force and mechanical force. The two lines can act antagonistically. Alternative configurations may also be used. For example, if a restorative force is used within a hydraulic mechanical mechanism, like a spring, then a single hydraulic line may be used. As shown in FIG. 27C, a spring may be used for biasing force instead of a pneumatic or hydraulic line. In this configuration, springs are used for the pressure in the hydraulic line. A motor (of the powered actuation system 132) can be used to compensate for the linear increase/decrease in force from the spring as the piston moves from the central position, turning the springs influence on the system into a constant force, rather than one that changes with displacement.

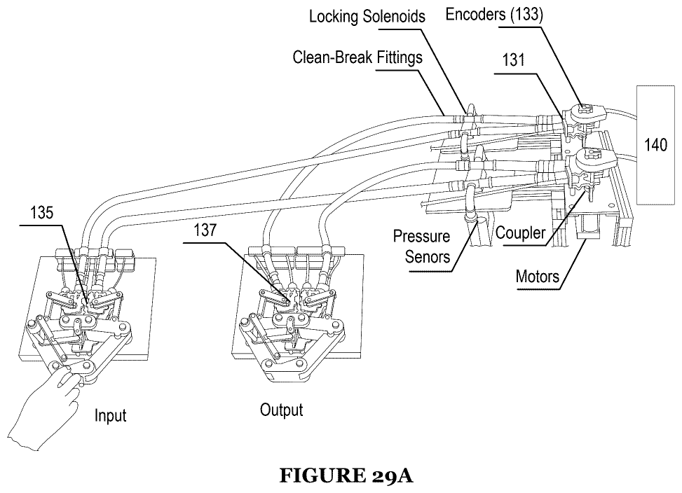

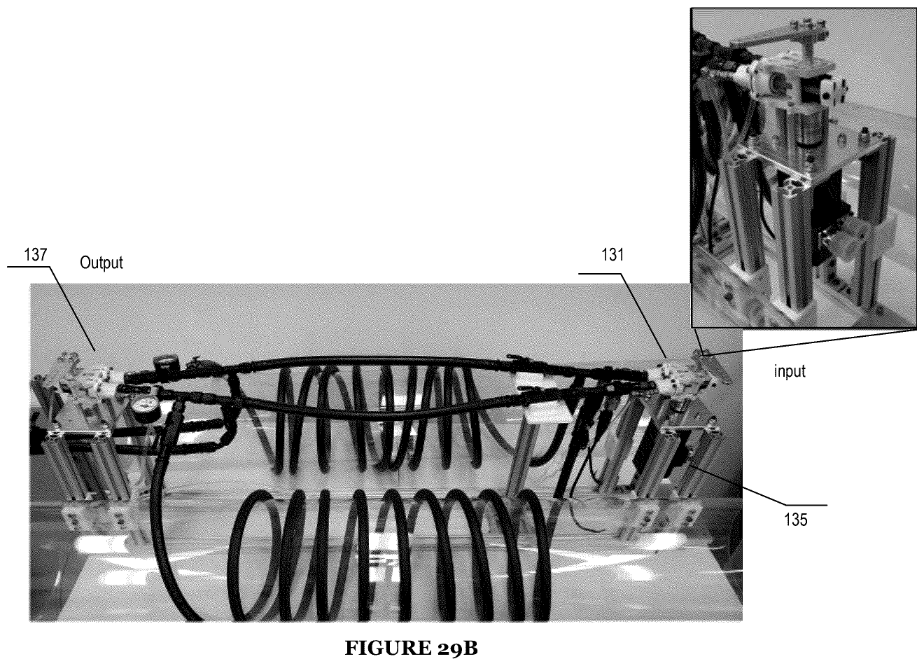

As shown in FIG. 29A and FIG. 29B, an exemplary powered and sensor-enabled transmission interface 131 can be implemented as an intermediary element between an input and output manipulator system. FIG. 29A show illustrative benchtop demonstration of how an input manipulator system 110 and an output manipulator system 120 may be integrated in a symmetrical manner. As shown in FIG. 29A, the system may include an input and output manipulator, integrated through a hydraulic transmission system 130. The hydraulic transmission system may include hydraulic lines with integrated locking solenoids (e.g., usable to lock DoF), clean-break fittings (e.g., for ease of modular adjustments and reconfiguration), motors and couplers used by a powered actuation system 132 to optionally augment or intervene with the actuation of the active transmission interface, pressure sensors, encodes, and a connected control system 140. The control system may integrate motor drivers, power supply, and/or other components used to drive the powered actuation system 132 or any other electrically controlled component.

In a similar variation shown in FIG. 29B, an active transmission interface may be mechanically coupled to an input, which may reduce the hydraulic lines to a single leg of hydraulic transmission.

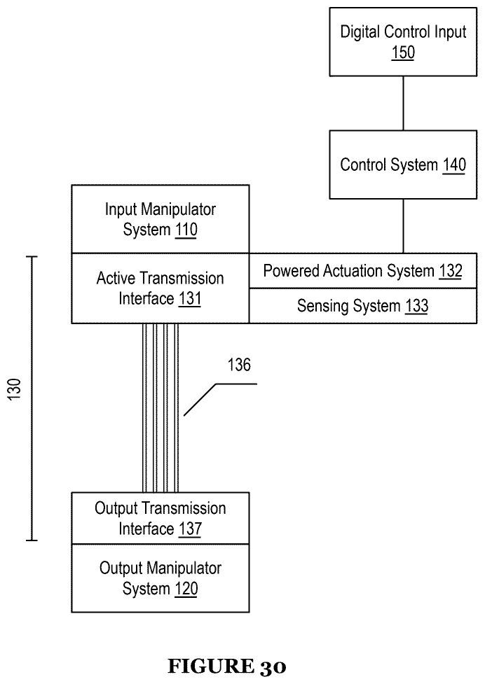

In addition to working with input manipulator systems coupled to the system for direct actuation/haptics, the system may additionally include additional inputs which could be a digital user control input and/or some automated control input. Accordingly, in some variations the system may additionally include a digital control input 150 as shown in FIG. 30. The digital input may be used to dynamically augment or supplement input from the input manipulator system 110. Accordingly, the output of the intermediary active transmission 131 may be based on a combination of the input received from the input manipulator system and the additional digital control input 150. The digital control input 150 may be a communicated digitally to the control system and/or the powered actuation system 132/active transmission interface 131. In some cases, the digital control input 150 may be connected via a network and could be a control input supplied from any network connected device. In some modes, the digital control input 150 may be used to control one or more degrees of freedom and/or all actuation of the output manipulator system 120.

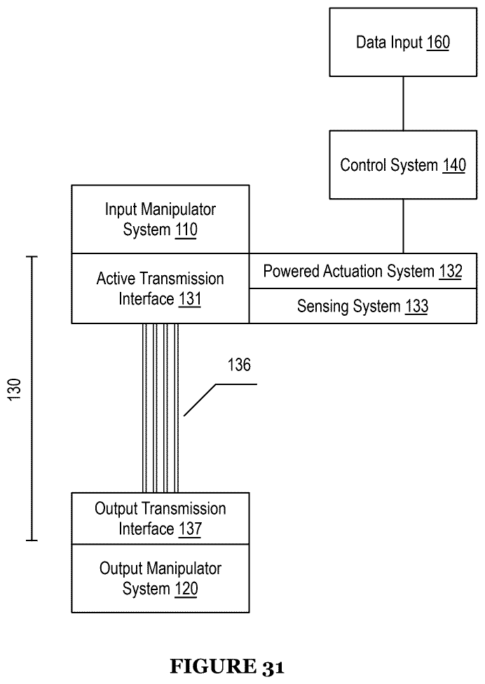

In a similar variation, the system may additionally include data inputs 160 or more specifically one or more types of medical data input as shown in FIG. 31. The control system may control the powered actuation system 132 (or otherwise control the active transmission interface 131) at least partially based on the data inputs. Data inputs 160 may include scanner data from the medical scanner or other sensor data collected and/or processed outside the system (e.g., MRI imaging data, CT scanner data, respiratory rate, heart rate, etc.). The control system 140 may additionally process or analyze the medical data input. The control system 140 may use data from the data input 160 to augment actuation in any suitable manner such as triggering haptic feedback based on the medical data input. For example, the system could simulate increased resistance when a device controlled by the output manipulator system 120 is approaching a detected “out of bounds” region. In another example, the system may high-pass filter or otherwise smooth actuation when in a certain area to avoid any slight tremors or accidental quick motions applied to the input manipulator system 110.

The input manipulator system 110 functions as the component or set of components used by an operator to control the output. The input manipulator systems 110 may be a single system used as a manipulator, which may alternatively be characterized as an actuator, end effector, or input device. The input manipulator system 110 is preferably a haptic controller that has direct physical coupling to the system such that force applied to the input manipulator system translates to force translated through the system and similarly forces be translated through the hydraulic transmission system 130 to the input manipulator system 110 as haptic feedback.

The input manipulator system 110 and similarly the output manipulator 120 may include a set of different levers, pulley systems, sliders, rotators, linkage systems, and/or other mechanisms that can be manipulated and actuated in a way that translates to actuation within the hydraulic transmission system 130.

The input manipulator system 110 can provide a configured set of degrees of freedom (DoF) used to direct actuation input into the system. The manipulator may use various mechanisms and/or linkage systems for offering different DoF. In one variation, the input manipulator system 110 can have six DoF of movement to enable full translational and rotational control of an output manipulator. In some variations, the input manipulator system 110 can include additional input elements with their own DoFs or input mechanisms such that other actions may be performed. For example, input mechanisms may be integrated into the input manipulator so that a needle or probe can be inserted or axially rotated.

Each DoF of the input manipulator system 110 may be coupled directly or indirectly to the hydraulic transmission system 130 and more specifically to the active transmission interface 131. In other words, a dedicated channel of the hydraulic transmission system may interface with each degree of freedom of an input manipulator system 110. In one variation, the input manipulator system 110 is mechanically coupled to an input of the active transmission interface 131. As such there may, in some variations, be no hydraulic actuation on the “input side” of the system. In another variation, the input manipulator system 110 may mechanically couple to a set of input hydraulic lines 134 through an input transmission interface 135. In this variation, the input manipulator system 110 has actuation conveyed through a hydraulic system to the active transmission interface 131, where it may then be conveyed on (possibly in an augmented manner) to the output manipulator system 120.

Haptic feedback is preferably delivered through the mechanical resistance or force applied from the hydraulic transmission system back to the input manipulator system 110.

The controlled DoFs of the input manipulator system 110 are generally described herein as being controlled through manipulation of a mechanical mechanism. In some variations, one or more DoFs may not be a mechanical input but could be fully or optionally controlled electronically. For example, one particular mechanical motion may be controlled using digital control from a computer or an electronic user interface.

The input manipulator may be formed or shaped with ergonomic considerations of the intended user.

In some variations, the input manipulator system 110 is the same, mirrors, or is substantially similar to the output manipulator system 120. However, the form and design of the input manipulator system 110 may vary in any suitable way or even be a distinct design from the output manipulator system 120.

The input manipulator system 110, in some variations, may be used in a less restrictive environment. As such, the input manipulator system 110 may be designed with fewer restrictions/limitations in the materials and types of components used in comparison with the output manipulator system 120.

The output manipulator system 120 functions as the one or more end effector where the intended output actions are performed. The output manipulator system 120, when used in a medical setting, is preferably the component used to perform the actions to a patient.

When the system is to be used alongside imaging technology like MRI, then the output manipulator system 120 may be made of compatible materials such as using plastic or other non-metal components. For example, the output manipulator system 120 may be made of materials compatible with an MRI machine or other type of scanner device. Scanner compatible materials may include non-magnetic materials, non-conductive materials, radio frequency (RF) transparent materials, non-ferromagnetic materials, and/or other material types that would be suitable for use in environments or conditions with material restrictions. This may additionally mean that system components in the restricted environment may lack any electronics.

The output manipulator system 120 may be mounted or positioned within a scanning region of a medical scanning device. For example, during use, the output manipulator system 120 may be oriented within a bore of an operating MRI machine.

Reference to the output manipulator system 120 as an output is used to primarily characterize its general role as the component used to perform a desired action. Force can similarly be conveyed from the output manipulator system 120 back to the hydraulic transmission system 130 and back (possibly in an augmented form) to the input manipulator system 110. Physical feedback imparted onto the output manipulator is preferably translated back as haptic feedback to the input manipulator system 110.

The hydraulic transmission system 130 functions to relay actuation between input manipulator system 110 and the output manipulator system 120. More specifically, the hydraulic transmission system 130 uses a set of hydraulic lines to convey actuation over some distance such that the input manipulator system 110 and the output manipulator system 120 may be separated by some distance. This distance of separation may be of arbitrary length but generally could range from 3 to 20 or more feet. There is preferably at least one hydraulic line for each channel (or DoF).

In one variation, the hydraulic transmission system 130 includes an intermediary active transmission interface 131 between the input and output manipulator systems 110 and 120, with at least one segment of hydraulic lines used to convey actuation. The hydraulic transmission system 130 may include hydraulic lines, hydraulic transmission interfaces, and optionally a powered actuation system 132 and/or a sensing system 133. The hydraulic transmission system 130 can be a serial network of hydraulic lines and transmission interfaces (e.g., hydraulic-to mechanical transmission interfaces), with optionally integrated powered actuation, sensing, and/or control components. The hydraulic transmission system 130 can include multiple channels to support transmission of multiple degrees of freedom.

Reference to a hydraulic transmission system 130 is used to describe a set of integrated subcomponents. The subcomponents of the system may alternatively be separated, grouped, and/or described in any suitable manner, including referencing their interconnected nature without referencing the hydraulic transmission system.

Accordingly, in one variation, the hydraulic transmission system 130 (or the system more generally) may include an intermediary active transmission interface 131 serially integrated between the input manipulator system 110 and the output manipulator system 120, an active transmission interface 131 comprising a powered actuation system 132 and/or a sensing system 133, an output transmission interface 137 that is coupled to the output manipulator system 120, and a set of hydraulic lines. The set of hydraulic lines preferably includes a set of output hydraulic lines 136 connecting to the intermediary active transmission interface 131 and the output transmission interface 137.

When there are two legs of hydraulic lines, the system may additionally include an input transmission interface 135 that is coupled to the input manipulator system, and additionally include a set of input hydraulic lines 134 connecting to the intermediary active transmission interface 131 and the input transmission interface 135. In this way, a hydraulic line may be established between the input and output with the active transmission interface being used to integrate a powered system and/or a sensing system.

The set of hydraulic lines functions to transmit hydraulic power from one component of the system to another. The hydraulic lines can transmit power thereby conveying actuation and/or force through the hydraulic lines. The system can convert pressure of hydraulic fluid in the hydraulic lines to kinetic energy to actuate a mechanical mechanism. The hydraulic lines preferably terminate with a transmission interface (e.g., a hydraulic-to-mechanical transmission system) to convert between hydraulic actuation and mechanical actuation.

The Hydraulic lines may be flexible or rigid. Flexible hydraulic lines may provide flexibility of use when used in a medical setting where equipment may need to be moved and adjusted. Combinations of flexible sections with rigid sections of hydraulic lines may also be used.

There is preferably a hydraulic line for each independent degree of freedom in a connected manipulator system. In some variations, the input manipulator system 110 and the output manipulator system 120 include corresponding numbers of DoF. There can be a hydraulic line for each channel or degree of freedom (e.g., one output hydraulic line and one input hydraulic line).

These distinct lines may make each DoF individually controllable such that each DoF may be controlled in different, independent ways. For example, a first DoF may be set to a passive mode where actuation is not augmented, while a second DoF may be augmented in a way that uses the powered actuation system to alter input from the input manipulator system 110 for the second DoF, a third DoF may be fully automated, and a fourth DoF may be locked to prevent actuation on that DoF. The modes of the different DoFs can also change independently. Accordingly, the control system can control the powered actuation system to augment hydraulic actuation translated through the active transmission interface for at least a subset of the degrees of freedom independently.

As discussed, the system preferably includes a set of output hydraulic lines 136. In some variations, the system may additionally include a set of input hydraulic lines 134. These different legs of hydraulic lines may be substantially similar. Hydraulic lines preferably are of scanner compatible materials in a restricted region. For example, at least the set of output hydraulic lines may be made of MRI compatible materials.

In some variations, the system may include a set of clean break fittings, which function to enable hydraulic lines to be connected and disconnected without pressure loss in the system. The clean break fittings can be integrated into the hydraulic line segments so that they can connect and disconnect on one or both ends. The clean break fittings may be used in proximity to the input or output manipulator systems 110 and 120 so that the manipulators can be changed. The clean break fittings may additionally or alternatively be used in proximity to the active transmission interface 131. In some cases, the components of the system can be reconfigured depending on the intended use of the system. For example, a system with a powered active transmission system can be reconfigured to be a fully passive system by reconfiguring the connections of the hydraulic lines to not include the powered active transmission system.

In some variations, the system may include one or more locking valves integrated into one or more hydraulic line. The locking valve may function as a physical brake to lock hydraulic actuation. There could be locking valves on one or more hydraulic lines so that one or more DoFs may be fixed based on if the locking valve is engaged. In one exemplary implementation, the locking valve is a solenoid valve that when engaged prevents actuation of the hydraulic line. Other types of locking valve mechanisms may alternatively be used. In some variations, a locking valve is a manually engaged mechanism, where a user must physically engage or disengage the valve. In some variations, a locking valve is an electronically controlled device, where the state of the valve can be digitally controlled.

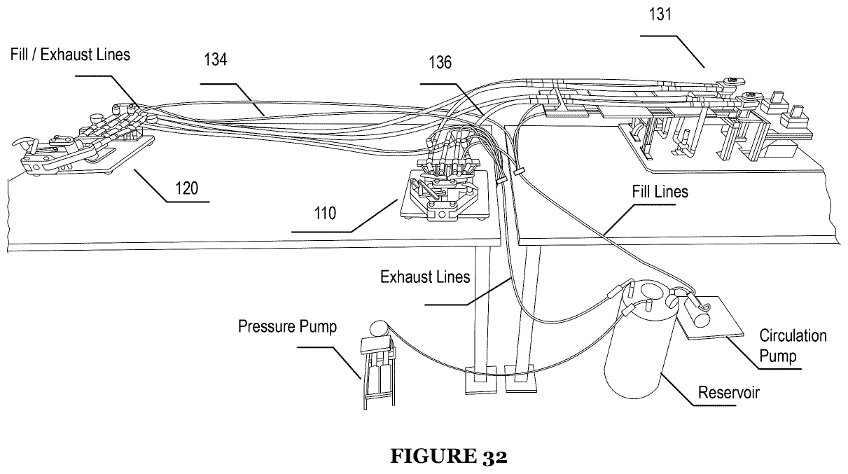

The system may include other hydraulic system sub-components like a reservoir, pressure pump, circulation pump, pressure sensor, fill/exhaust lines and valves, and/or other components as shown in FIG. 32. Fill lines and the exhaust lines may connect to the components of the system like the transmission interfaces, as shown in FIG. 32. In FIG. 32 not all hydraulic/pneumatic connections are shown. Alternative configurations and interconnections may alternatively be used. Similarly, subcomponents such as those described above may include features for use with a hydraulic system. In one variation, the transmission interface can include a fill port, where hydraulic fluid or gas can be added or removed from the system as shown in FIG. 28A and FIG. 28B.

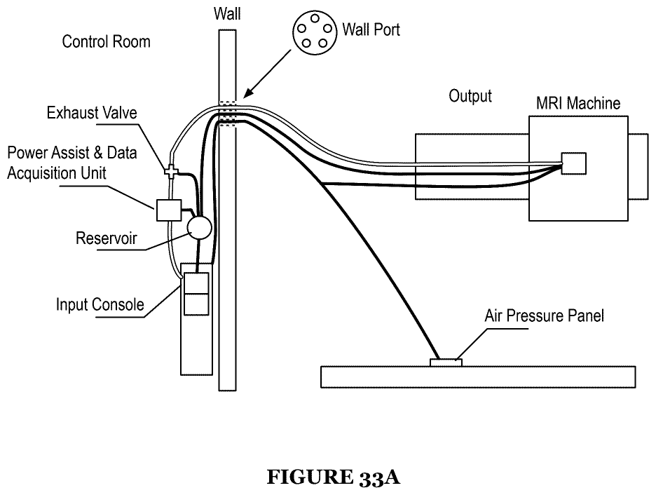

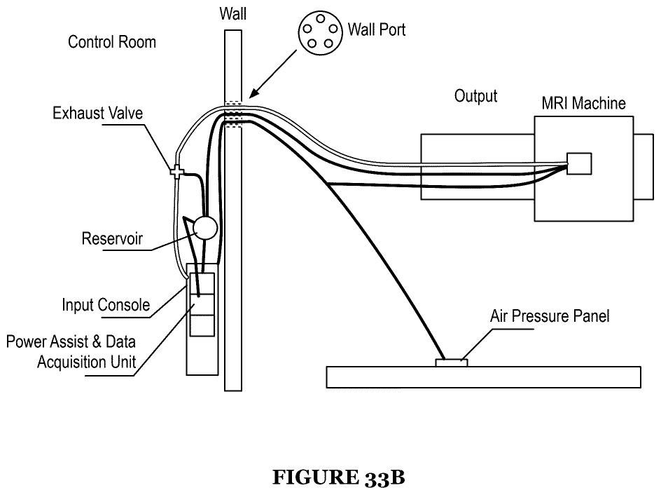

In some variations, a subset of the system components may be arranged on one-side of a physical barrier. As shown in FIGS. 33A and 33B, components like the active transmission interface 131, an input console, hydraulic subsystems (e.g., reservoirs and exhaust valves) may be arranged on a first side of a barrier (e.g., a shielded unrestricted environment), and the output transmission system and output manipulator systems can be arranged on a second side of the barrier (e.g., a restricted environment near a medical scanner). FIG. 33A shows a variation with input and output hydraulic lines. FIG. 33B shows a variation with only output hydraulic lines. This functions to isolate any of the restricted components from the second side (which is where a device like an MRI or CT machine may be used). The input console may include user input device, monitors, visualization system, digital controls, interfaces with external data inputs, and/or other components.

A transmission interface functions to convert between hydraulic actuation and another form of actuation (e.g., mechanical actuation). Transmission interfaces may be used to on the input and output side to translate from hydraulic actuation from a hydraulic line to mechanical actuation of a manipulator system. Transmission interfaces in the case of the active transmission interface 131 may additionally expose a mechanism by which powered actuation and/or sensing may be applied.

As shown in FIG. 25 and FIG. 27, transmission interfaces (active and non-active) and hydraulic lines may be connected in series for one degree of freedom of actuation. Multiple parallel channels may be established and used for controlling multiple DoF. In some instances, the transmission interface used for the input (135) and/or output (137) may be designed with a mechanism in a similar manner to the active transmission interface 131 described here and may be implemented without sensing or powered actuation.

The transmission interface may use two integrated hydraulic-to-mechanical transmission components such that hydraulic actuation and mechanical actuation are linked. In one variation, a transmission interface, include two coupled hydraulic-to-mechanical mechanisms such that, as a whole, the active transmission interface 131 translates from hydraulic actuation to mechanical actuation and back to hydraulic actuation.

In one variation, a transmission interface uses a hydraulic actuator with rolling diaphragm to translate external motion (e.g., linear actuation) to hydraulic actuation within a hydraulic line. Use of a rolling diaphragm may experience reduced friction and smoother actuation. Alternatively, other types of mechanisms may be used to translate external actuation to hydraulic actuation. In one variation, a transmission interface used for the input and/or output manipulator systems can have a rotational actuator (e.g., gear, cable, belt) engage with two linear gears moving in opposing/inverse directions to create inverse hydraulic actuation in two hydraulic lines. A gearing system may be used to alter the translation of hydraulic actuation through the transmission interface.

As shown in FIG. 28A, a transmission line with an input transmission interface 135, an intermediary active transmission interface 131, and an output transmission interface 137 with similar mechanical designs can be connected to form a full hydraulic line/channel. When in a passive mode, actuation at the input or output will translate to the opposing end. When in a controlled augmented mode, a motor of a powered actuation system 132 attached or otherwise coupled to a shaft of a rotational element of the transmission system (as shown in the example of FIG. 28A) may be used to augment actuation. As discussed, an alternative topology may be used whereby the system can use one inactive transmission interface for coupling to a manipulator and an active transmission system for perturbing actuation. As shown in FIG. 28B, a transmission line may include: an active transmission interface 131 coupled mechanically to an input, an output transmission interface 136, and output hydraulic lines integrating the two transmission interfaces.

As the output transmission interface 137 may be used in a restricted region, in some variations, the transmission interfaces or at least the output transmission interface 137 may be made of compatible materials.

The active transmission interface 131 functions to supplement and/or augment the hydraulic actuation between the input manipulator 110 and the output manipulator 120. The active transmission interface 131 allows sensing and/or actuation augmentation to be used within the hydraulic transmission system 130.

The active transmission interface 131 is an intermediary component that is serially integrated between the input manipulator system 110 and the output manipulator system 120. The active transmission interface 131 may include or be connect to a powered actuation system 132 such that the active transmission interface 131 is a powered active transmission interface 131. Additionally or alternatively, the active transmission interface 131 may include a sensing system 133 such that the active transmission interface 131 is a sensor-enabled active transmission interface 131. The powered actuation system 132 and sensing system 133 may be used in combination to make a sensor-enabled and powered active transmission interface 131.

As in the non-active transmission interfaces, the active transmission interface 131, in one variation, may include two coupled hydraulic-to-mechanical mechanisms such that, as a whole, translates from hydraulic actuation on one interface (e.g., an input) to mechanical actuation and back to hydraulic actuation at another interface (an output). The mechanical actuation portion may be augmented with a powered actuation system and/or be sensed with a sensing system.

In one variation, the two hydraulic-to-mechanical mechanisms are mechanically coupled together. In one example, the exemplary hydraulic-to-mechanical mechanisms can both engage with a rotary gear such that their actuation is coupled. A motor mechanically coupled to the rotary gear could be actively engaged to amplify or resist actuation. The powered actuation system 132 may have a motor directly or indirectly (e.g., through a gearing system) to a shaft extending from the rotary gear. A sensing system 133 may additionally or alternatively integrate with the rotary gear to sense the actuation transferred through the hydraulic transmission system 130. For example, the rotation of the gear may be sensed using an encoder or other sensing mechanism.

Other mechanisms may alternatively be used to function as a hydraulic transmission interface with integrated powered actuation and/or sensing. In one exemplary variation, an alternative transmission mechanism may be used so that two hydraulic-to-mechanical transmissions could be physically disconnected where an electronic or other control mechanism is used to sense and/or control the mechanical actuation of each portion of the interface. This may be used so that all or part of the system could be remotely controlled.

Alternatively, if used on the input transmission interface, then the active transmission interface may translate mechanical actuation of the input manipulator to hydraulic actuation.

The powered actuation system 132 functions to augment the actuation occurring within the hydraulic transmission system 130. In one variation, the powered actuation system 132 includes a motor that mechanically couples to a hydraulic-to-mechanical transmission mechanism. The motor may couple to a rotary gear of a hydraulic-to-mechanical transmission through a motor attachment shaft and/or using a gear train. The powered actuation system 132 may use a backdrivable motor that couples with a rotary gear of the hydraulic-to-mechanical transmission mechanism such that it can allow rotation without being driven. The powered actuation system 132 may include other components used in the operation and control of a motor-powered system such as motor encoders, motor drivers, power systems, control systems, and/or other suitable components.

The powered actuation system 132 may include one motor for each channel and controlled DoF. Each motor can be independently controlled.

The powered actuation system is preferably controlled by a control system 140. The control system may manage how actuation is translated through the system. The powered actuation system 132 may be used to provide resistive forces or supportive forces. The powered actuation system 132 can additionally disengage to allow actuation to occur passively. The powered actuation system may be controlled based on actuation from the input manipulator system 110 and/or the output transmission interface 137, and optionally from input from the sensing system 133, digital control inputs 150 and/or supplemental data input.

The sensing system 133 functions to collect data during use of the device. The sensing system 133 can preferably track the actuation of the system. In one variation, the sensing system 133 can provide data that is used as input to a control system 140 of the powered actuation system 132. In this way, the system can respond to how a user is manipulating the input manipulator system 110 and/or the haptic feedback from the output manipulator system 120. In another variation, actuation data may be collected in coordination with medical data from the same time. This can be used to train data models used to automate or otherwise augment actuation in later uses of the system. In some alternative variations, a sensor enabled active transmission interface 131 may be used independent of powered actuation. This non-powered actuation implementation may be a selectable mode if a fully passive mode is used but sensing and data collection is still desired.

The sensing system 133 may use encoders, pressure sensors, and/or other sensors to monitor state of the system that can be used for determining how to control the powered actuation system 132. In one variation, the sensing system 133 may be integrated into the active transmission interface 131. For example, the sensing system may sense mechanical actuation of a transmission interface. In one exemplary variation, an encoder coupled to a rotary gear of a hydraulic transmission interface may be used to track actuation applied between two coupled hydraulic lines.

In another variation, a sensing system 133 may be integrated into another part of the system or even be data provided by an outside system. For example, a sensing system 133 could be hydraulic sensing system integrated into some part of the hydraulic transmission system 130. This may include measuring pressure and/or actuation within a hydraulic line. In one variation, pressure may be measured on two legs of the hydraulic transmission system 130—measuring pressure from an input hydraulic line and from an output hydraulic line. A pressure differential calculated from the sensed pressures may be used to do friction compensation and/or inertia compensation. Other sensors may alternatively be used.

The system may additionally or alternatively include one or more additional data input 160, which functions as a supplemental data source that can be used within the system. The data input 160 can additionally or alternatively be used to control a powered actuation system 132. Such a supplemental data input 160 may be a medical data input 160. For example, data from an active scanner device (e.g., an MRI machine or CT scanner) could be used to dynamically control, at least in part, the powered actuation system 132.

The powered actuation system 132 may be used in a variety of ways. In one variation, the powered actuation system 132 may be used in one of a set of modes such as a passive mode, power-assist mode, and/or a fully autonomous mode. The powered actuation system 132 could dynamically switch between such modes or alternatively be configured to always operate in one of the described modes.

A control system 140 functions to manage augmentation of actuation. The control system can include configuration or otherwise be configured to control the powered actuation system to at least partially augment hydraulic actuation (e.g., the haptic interactions) translated through the active transmission interface 131 between the input manipulator system and the output manipulator system. The control system 140 may be communicatively coupled to and integrated with the powered actuation system 132, such that the control system 140 can manage augmentation of actuation applied by the powered actuation system 132 to the active transmission interface 131.

The control system 140 may include one or more processors and one or more computer-readable mediums (e.g., non-transitory computer-readable mediums) storing instructions that, when executed by the one or more processors, cause the control system 140 to perform operations related to controlling actuation translated through the active transmission interface 131. The control system 140 may alternatively be configured as a hardware implementation where the control system 140 includes an electronic control system. Both variations may include configuration or be configured to control the powered actuation system to at least partially augment hydraulic actuation translated through the active transmission interface between the input manipulator system and the output manipulator system. The operations may vary depending on the implementation and state.

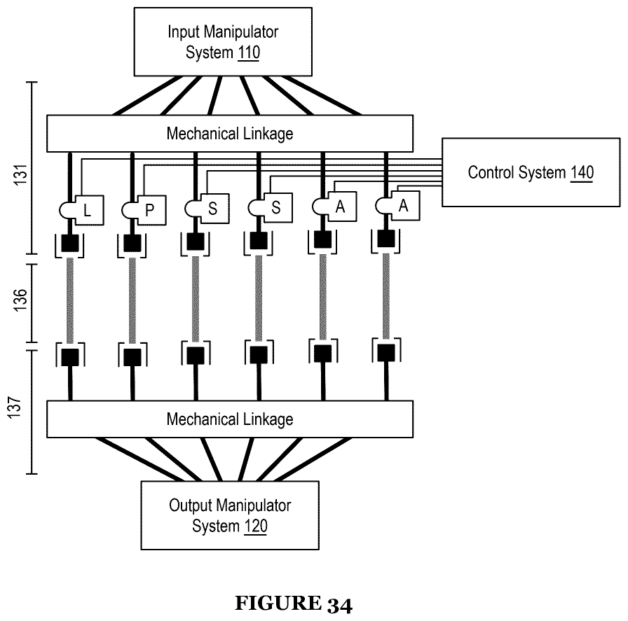

In one variation, the control system 140 may include configuration to translate hydraulic actuation through the active transmission interface 131 in one of a set of modes, where at least one mode of the set of modes is a mode that augments hydraulic actuation. This may include different distinct modes or dynamic modes that adjust augmentation in real-time. The various modes may include a passive mode, a power-assist mode, and/or an automated mode. Different degrees of freedom may be individually controlled with dynamic control. As shown in FIG. 34, a system with six DoF may have the different DoF in different states where the powered actuation system 132 is controlled such that: a first motor is in a locked state (L), a second motor is in a passive state (P), a third and fourth motor are in power-assist or semi-automated mode(S), and a fifth and sixth motors are in an automated state (A). This establishes the different channels in a locked state, passive state, semi-automated state (e.g., a power assist mode) or in an automated state. Additionally, the set of different channels may be controlled in a coordinated manner.

In a passive mode, the powered actuation system 132 may be inactive and rotate freely such that the output actuation is a direct result of input actuation. When configured to apply a passive mode for one or more DoF, the control system 140 may deactivate a motor of a relevant transmission interface 131. Accordingly, in a passive mode, the powered actuation system 132 (and more specifically a motor of the powered actuation system 132) can be unpowered/unenergized. The motor of the powered actuation system 132 may remain mechanically engaged and coupled to the active transmission interface, but may not apply powered actuation. The motor of the powered actuation system 132 can be backdrivable so that it can be rotated freely by user control at the input 110.

In an alternative implementation, when in a passive mode, a motor of the powered actuation system 132 may be mechanically disengaged from the active transmission interface 131. Being mechanically disengaged may mean that the motor is mechanically decoupled and unconnected from some mechanism to drive or intervene with actuation. A clutch may be used to facilitate mechanical disengagement. Use of a clutch in a passive mode, may allow the motors to potentially expand viable motor boxes such as non-backdrivable motors.

In a power-assist mode (i.e., a “semi-automated” mode), the powered actuation system 132 may intervene, augment, or otherwise alter actuation supplied by the input. When in a power-assist mode, hydraulic actuation input from the input manipulation system is translated by a function to hydraulic actuation output delivered to the output manipulation system. In this way actuation is the combination of actuation or force at the input manipulator system 110 and/or the output manipulator system 120 with the actuation force from the powered actuation system 132 at the active transmission interface 131.

A power-assist mode may be implemented with one or more different features to assist or otherwise alter the actuation control and/or haptic feedback. A power assist mode may be used in various ways. In some examples power assist may be used to filter or otherwise adjust input actuation, compensate for various aspects of operation, and/or alter or enhance haptic feedback.

In a power assist mode variation for filtering input, a power-assist mode may be used for noise removal or signal smoothing. This may, for example, be used to remove a user's input tremors from transferring to the output. A lowpass filter on the actuation may be used to prevent sudden jerks of motion.

In a power assist mode variation for compensation, the powered actuation system 132 may be used to compensate for friction and/or inertia within the system. This can be used to remove the appearance of friction or inertia to the operator of the systems. Configuration may include sensing friction or inertia and then applying counteractive control to the powered actuation system 132 to counter the friction or inertia. Sensing friction or inertia may include sensing pressure differentials between an input transmission line and an output transmission line.

In a power assist mode for haptic feedback, configuration controls the powered actuation system 132 to alter haptic feedback experienced by the input manipulator system. Haptic feedback augmentation may be used to amplify (or alternatively deamplify) haptic feedback transferred from the output back to the input. In some variations, such haptic feedback augmentation could be based on externally sensed input(s). Sensed inputs may be based on the sensed state of the subject, sensed state of the operator, and/or sensed state from an imaging device. For example, the position of an operating tool in relation to a region of interest in the patient, as determined through MRI imaging or other scanner data, could be used to artificially supply haptic feedback to the operator.

In another variation, other input effects could be implemented through dynamic control of the power assist mode. For example, added or removed hysteresis in movement resistance could be enabled by the powered actuation system 132.

In a fully autonomous mode, the powered actuation system 132 may operate without input supplied by the input manipulator system 110. In other words, in an automated mode, hydraulic actuation may be driven entirely by the intermediary active transmission 131 using the powered actuation system 130. The fully autonomous mode may be used for fully autonomous procedures or steps. Alternatively, the autonomous mode may be used for select actions. For example, an autonomous mode may be used to move an output manipulator (e.g., an operating tool) into position before enabling manual control. In another example, a subset of DoF may be in an autonomous mode. For example, a doctor may move a tool into a position and then lock the position of tool relative to the medical imaging. The tool may track the position of the tool relative to the medical imaging so that it stays aligned to the point of the body. In another example, select actions may be performed using an autonomous mode. Such autonomous modes may be controlled based on historical data collected through previous operation of similar systems.

### 6. Hydraulic System Methods

A method for a hydraulic transmission system of a teleoperation device with haptic feedback can include controlling a system such as described above to provided augmented actuation. The method functions to use a hydraulic transmission to alter hydraulic actuation and feedback. The method is preferably implemented through the operation of a hydraulic manipulator. The method is preferably used in combination with a system variation as described herein but may alternatively be used with alternative system implementations.

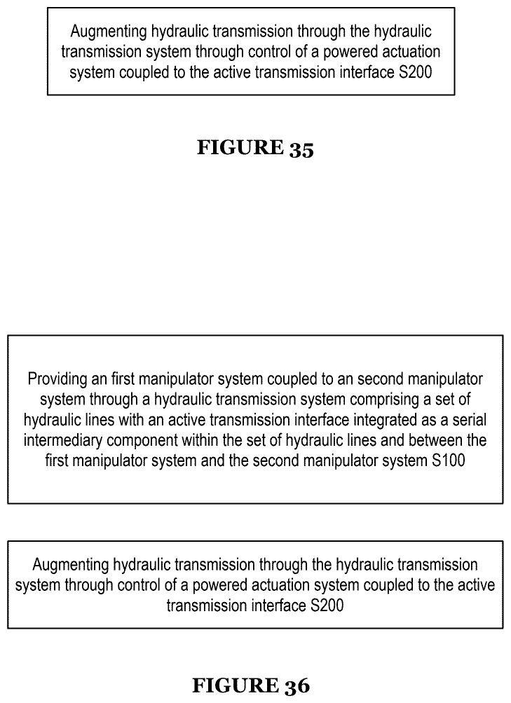

The method may include, at a system such as described above with a hydraulic transmission system with an active transmission interface as an intermediary component between an input manipulator system and an output manipulator system, augmenting hydraulic transmission through the hydraulic transmission system through control of a powered actuation system coupled to the active transmission interface S200 as shown in FIG. 35.

The method may additionally include assembling, configuring, or otherwise providing the system. Accordingly, as shown in FIG. 36, the system may include providing an first manipulator system coupled to an second manipulator system through a hydraulic transmission system comprising a set of hydraulic lines with an active transmission interface integrated as a serial intermediary component within the set of hydraulic lines and between the first manipulator system and the second manipulator system S100, and augmenting hydraulic transmission through the hydraulic transmission system through control of a powered actuation system coupled to the active transmission interface S200. The first manipulator system may be an input manipulator system and the second manipulator system may be an output manipulator system. The method may additionally or alternatively be used to transfer from an output device to an input device as such the first manipulator system may be an output manipulator system and the second manipulator system may be an input manipulator system.

In some variations, the method may be implemented in connection with a non-transitory computer-readable medium storing instructions that, when executed by one or more computer processors of a computing platform, cause the computing platform to perform operations comprising: providing an input manipulator system coupled to an output manipulator system through a hydraulic transmission system comprising a set of hydraulic lines with an active transmission interface integrated as a serial intermediary component within the set of hydraulic lines and between the input manipulator system and the output manipulator system S100, and augmenting hydraulic transmission through the hydraulic transmission system through control of a powered actuation system coupled to the active transmission interface S200. The computer-readable medium may additionally include configuration for any suitable variation described herein.

In a similar way, the method may be implemented as part of a system such as a control system whereby the system includes one or more computer-readable mediums storing instructions that, when executed by the one or more computer processors, cause a computing platform to perform operations comprising: augmenting hydraulic transmission through the hydraulic transmission system through control of a powered actuation system coupled to the active transmission interface S200. The system may additionally include the components of the system described herein.

Block S100 functions to provide, assemble, configure, or otherwise perform operations in connection with a system as described herein. In some variations, the method may be performed with a system differing in some manner from the system described herein.

Block S100 may specifically include providing an input manipulator system coupled to an output manipulator system through a hydraulic transmission system comprising a set of hydraulic lines with an active transmission interface integrated as a serial intermediary component within the set of hydraulic lines and between the input manipulator system and the output manipulator system. The providing the system may include the producing or setting up of any of the components described herein such as input manipulator system(s), output manipulator system(s), a hydraulic transmission system, an active transmission interface, a powered actuation system, a sensing system, input hydraulic lines, input transmission interface, output hydraulic lines, output transmission interface, control system, a digital control input, and/or a data input.

In these method variations, the method may be applied with a system including any suitable hydraulic network topology.

In one variation, the method may be used with a hydraulic transmission system that includes a single output leg of hydraulic lines bridging a distance between the active transmission interface and an output transmission interface. In this variation, the input manipulator system may be directly coupled to the input of the active transmission interface through a linkage system; and translating the actuation input to actuation at the input of the active transmission interface may include translating the actuation input through mechanical actuation of the linkage system.

In another variation, the method may be applied with a hydraulic transmission system that includes an input leg and output leg of hydraulic lines. The input hydraulic lines bridge a distance between the active transmission interface and an input transmission interface. The output hydraulic lines bridge a distance between the active transmission interface and an output transmission interface. In this variation, the input manipulator system is coupled to the input of the active transmission interface through a set of input hydraulic lines of the set of hydraulic lines; and translating the actuation input to actuation at the input of the active transmission interface includes translating the actuation input from the input manipulator system through the set of input hydraulic lines as hydraulic actuation to active transmission interface.

Block S200, which includes augmenting hydraulic transmission through the hydraulic transmission system through control of a powered actuation system coupled to the active transmission interface, functions to control how actuation, force, and/or pressure is conveyed within the hydraulic transmission system.

Augmenting hydraulic transmission through the hydraulic transmission system is used to dynamically adjust how an input to an input manipulator and/or an output at an output manipulator is experienced and/or expressed. In some variations, augmenting hydraulic transmission through the hydraulic transmission system may include affecting manipulation at the input manipulator system, affecting manipulation at the output manipulator system, translating manipulation at the input and the output manipulator systems through a hydraulic system that is configured to have an active transmission interface as a serial intermediary component between the input manipulator system and the output manipulator system, and augmenting hydraulic transmission through the active hydraulic transmission system by controlling a powered actuation system coupled to the active transmission interface.

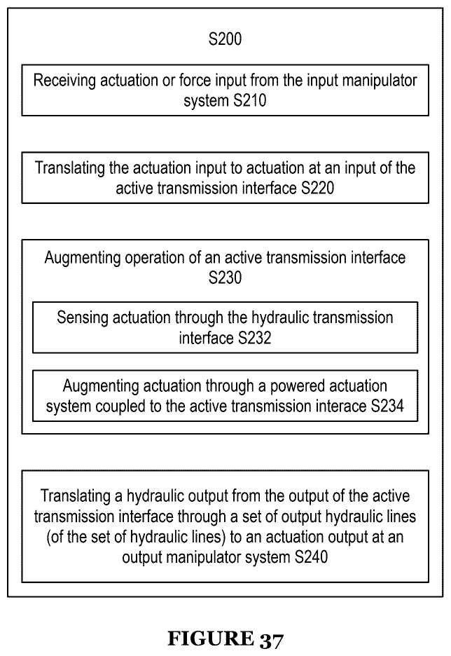

When described how it may be expressed when a user applies some input, augmenting hydraulic transmission through the hydraulic transmission system may include, as shown in FIG. 37: receiving actuation or force input from the input manipulator system S210; translating the actuation input to actuation at an input of the active transmission interface S220; augmenting operation of a hydraulic transmission system S230; and translating a hydraulic output from the output of the active transmission interface through a set of output hydraulic lines (of the set of hydraulic lines) to an actuation output at an output manipulator system S240.

Block S210, which includes receiving actuation or force input from the input manipulator system, functions to receive input of an operator. The input is preferably implemented through mechanical manipulation of some mechanism with a number of mechanical DoFs. Haptic feedback is preferably also experienced at the input manipulator. In some cases, other input mechanisms may be used in the input device.

In one variation, a single input manipulator is designed to receive the manual input of one operator. In other variations, multiple input manipulators may be used to deliver multiple inputs. For example, for certain types of operations, multiple operator tools or tools with multiple controllable elements may be used. Multiple different operators (e.g., doctors) may supply input to control a subset of the elements. In some variations, one or more aspects may be controlled by an automated system.

In addition to receiving input from a manipulator system, the method may additionally receive input from a digital control input or another data input. A digital control input may be some input signal that is supplied from digital or electronic device. In one example, the method may be able to additionally handle receiving an input signal from a digital device over a network. This may function to allow teleoperation from practitioners far removed. For example, when used for a teleoperation, an expert practitioner could connect in to handle some aspects of an operation from a remote location (e.g., connected via a communication network), but a local practitioner could still use the input manipulator to have direct actuation control to assist or even take over should something happen with the remote connection.

Block S220, which includes translating the actuation input to actuation at an input of the active transmission interface, functions to relay the input from the input manipulator to a hydraulic transmission system. The hydraulic system transfers the input to actuation at an output. This transfer of input may or may not include some augmentation at given point of time. The use of a hydraulic system may be used so that the device can be used in environments where electronics and/or metal components are not feasible such as within an MRI device.

Block S240, which includes translating a hydraulic output from the output of the active transmission interface through a set of output hydraulic lines to an actuation output at an output manipulator system, functions to convey some output over a hydraulic line to an output manipulator system. This conveyed output is used to control execution of some action (e.g., motion or applied force) at some output mechanism. In some applications, an output manipulator may be situated in an environment where electronics and/or metal components may not be used. As such a portion of the hydraulic line to the output manipulator and the output manipulator may be made of compatible materials and components. For example, when used with an MRI device, the output manipulator may be made of plastic and non-metallic materials.

The output manipulator may mirror that of the input manipulator. However, the output manipulator may be of a different design and the output may be of a different actionable form from the input. For example, an angular DoF at the input may be used to control a linear DoF at the output.

In one variation, an input manipulator may be directly mechanically coupled to an interface of the active transmission interface. In another variation, the input manipulator may be indirectly coupled to the active transmission interface through a hydraulic connection (e.g., input hydraulic lines).

The method can support reconfiguration of the system to alter forms of actuation control. In one variation, the method can include reconfiguring one or more DoFs for passive control and/or augmented or autonomous control. For example, the hydraulic lines connections can be configured to route around an active transmission interface to be used in a passive mode. Similarly, the hydraulic lines can be configured to use different types of input and/or output manipulators. In another variation of reconfiguration, hydraulic brakes may be engaged or disengaged to selectively enable or disable select DoF.

Block S230, which includes augmenting operation of a hydraulic transmission system, functions to collect information on operation and/or to alter the actuation, force or pressure transmitted between the input and outputs of the hydraulic transmission system. This may be used to enable powered actuation and/or sensing and monitoring of actuation.

In one variation, augmenting the hydraulic actuation may additionally or alternatively include sensing actuation through the hydraulic transmission interface S232.

Additionally or alternatively, augmenting the hydraulic actuation in one variation includes augmenting actuation through a powered actuation system coupled to the active transmission interface S234. Augmenting the hydraulic actuation/force through the powered actuation system S234 may more specifically include dynamically controlling the powered actuation system coupled to the active transmission interface as actuation/force is translated between the input of the active transmission interface to an output of the active transmission interface.

Augmenting actuation/force through a powered actuation system S234 may include augmenting actuation transmitted through the hydraulic system in one of a set of modes. Exemplary modes may include a passive mode, power-assist mode, and/or an automated mode. Augmenting actuation/force through a powered actuation system may additionally include switching mode for one or more channels (DoF) of the hydraulic transmission system. Additionally, dynamically controlling the powered actuation system may additionally include dynamically controlling the powered actuation system independently for different subsets of channels. Each channel may include a line of transmission interfaces and hydraulic lines coupling to a degree of freedom of an input and output manipulator.

A passive mode functions to enable the system to operate, at least temporarily, without active augmentation. When in a passive mode, the hydraulic transmission system will generally function as hydraulic system to transmit actuation/force from an input to an output. A passive mode may be engaged for all DoF (thereby for each line of the hydraulic transmission system). A passive mode may alternatively be engaged for a subset of DoF (e.g., for one or more of the lines of the hydraulic transmission system). In a passive mode, the method may include transmitting actuation through the hydraulic system without augmentation. Augmenting the hydraulic actuation/force through an active hydraulic transmission interface may include, at a control system, disengaging or deactivating a motor of the powered actuation system coupled to an active transmission interface for each line set to a passive mode. If the full hydraulic transmission system is set to a passive state, then augmenting hydraulic actuation/force when in a passive mode can include disengage or deactivating each motor of the powered actuation system coupled to the active transmission interface.

A power-assist mode functions to alter actuation supplied by some input. The input supplied by an operator to the input manipulator can be altered by the powered actuation system when in a power-assist mode.

This may be used to alter the output actuation. This may additionally or alternatively be used to alter the haptic feedback experienced by the input manipulator. As discussed herein, power-assist modes can be used for various power-assist features such as reducing effects of friction or inertia, smoothing or filtering input, and/or altering actuation in other ways.

In some variations, the power-assist mode may be used in compensating for losses or deficiencies in the system. This may be used to counteract friction or inertia for example. In one variation, differential pressure sensing performed across the hydraulic lines as shown in FIGS. 28A and 28B, can be used to apply friction compensation and/or inertia compensation using the powered actuation system. In this variation, augmenting actuation/force through a powered actuation system can include sensing pressure differential and determining friction and/or inertia and applying, using the powered actuation system, a compensating force based on the friction or inertia.

In some variations, the power-assist mode may be used for filtering or changing how an input is translated to an output. In such a mode, augmenting actuation/force through a powered actuation system may include applying a filter to actuation/force from an input manipulator system. The filter could be a lowpass filter to eliminate sudden movements, jerk motions, and/or tremors. This may smooth out actuation at the output manipulator. Alternatively, any suitable transform or function may be used to translate an input force/actuation to an output force/actuation.

In some variations, the power assist mode may be used to alter input based one or more additional data input. The additional data input may be sensed data. The additional data input may additionally or alternatively be a medical data input. As mentioned, the method may include receiving an additional data input, and wherein augmenting actuation through a powered actuation system may include controlling input-output translation based on additional data input(s). An example of a medical data input may include scanner data from an MRI, CT scanner, heart rate, respiratory rate, and/or any suitable data related to medical conditions or conditions of a subject.

In some variations, the additional data input provides information on sensed or detected state of the output device. This may enable MRI imaging or other forms of monitoring to alter the controls of a device.

As the system enables use of a manipulator during live scanning with restrictive scanning regions, the method may enable novel operations using live real-time scanning data. For example, augmenting control may be used for controlling actions of an output manipulator system during live MRI scanning, wherein controlling the actions is based in part on MRI images resulting from the scanning.

For example, using MRI imaging, the powered active transmission interface may alter the power-assist mode based on where a tool is in relation to a particular region of the body. This may be used to prevent moving a tool into restricted regions, to alter the precision of the tool when in vicinity to a targeted region, and/or enable other operation features.

As another variation, a power assist mode may be used to enhance or generate haptic feedback. in a power assist mode used to enhance haptic feedback, augmenting actuation/force through the powered actuation system may include, at a powered actuation system, triggering a haptic feedback action to at least an input, wherein triggering is based on a detected condition. The detected condition may depend on additional data inputs. The haptic feedback may simulate a synthetic wall, barrier, region with “rough” feeling actuation (e.g., increased friction), a region with smooth actuation (e.g., decreased friction), vibration alerts, and/or other forms of haptic feedback.

An automated mode functions to control actuation entirely or primarily using the powered actuation system 132. An autonomous mode may be engaged for one or more degrees of freedom. In a fully automated mode, actuation is fully driven by the automated control of the powered actuation system. In some variations, a partial automated mode may enable input supplied from the input manipulator system to dynamically adjust or take over from the automated mode. For example, an automated mode may be engaged. For a time, a practitioner may let the automated mode perform its task, but if desired, the practitioner could begin to supply input to the input manipulator system and that could override the automated control while input is supplied. In some variations, input to the input manipulator system may deactivate the automated mode. In some variations, input will adjust the automated actions, but then automated control can restart immediately.

### 7. System Architecture

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, but any suitable dedicated hardware device can (alternatively or additionally) execute the instructions.

In one variation, a system comprising of one or more computer-readable mediums (e.g., non-transitory computer-readable mediums) storing instructions that, when executed by the one or more computer processors, cause a computing platform to perform operations comprising those of the system or method described herein such as: providing an input manipulator system coupled to an output manipulator system through a hydraulic transmission system comprising a set of hydraulic lines with an active transmission interface integrated as a serial intermediary component within the set of hydraulic lines and between the input manipulator system and the output manipulator system, and augmenting hydraulic transmission through the hydraulic transmission system through control of a powered actuation system coupled to the active transmission interface.

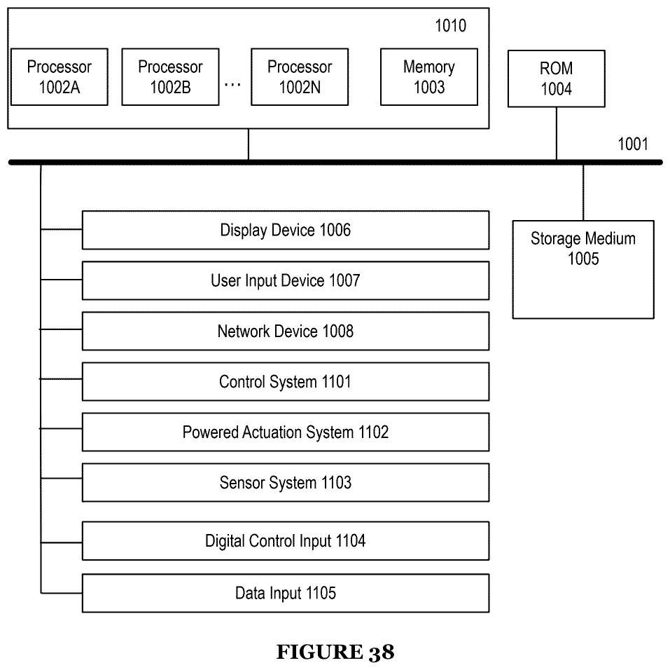

FIG. 38 is an exemplary computer architecture diagram of one implementation of the system. In some implementations, the system is implemented in a plurality of devices in communication over a communication channel and/or network. In some implementations, the elements of the system are implemented in separate computing devices. In some implementations, two or more of the system elements are implemented in same devices. The system and portions of the system may be integrated into a computing device or system that can serve as or within the system.

The communication channel 1001 interfaces with the processors 1002A-1002N, the memory (e.g., a random-access memory (RAM)) 1003, a read only memory (ROM) 1004, a processor-readable storage medium 1005, a display device 1006, a user input device 1007, and a network device 1008. As shown, the computer infrastructure may be used in connecting control system 1101, powered actuation system 1102, sensor system 1103, digital control input 1104, data input 1105, and/or other suitable computing devices.

The processors 1002A-1002N may take many forms, such CPUs (Central Processing Units), GPUs (Graphical Processing Units), microprocessors, ML/DL (Machine Learning/Deep Learning) processing units such as a Tensor Processing Unit, FPGA (Field Programmable Gate Arrays, custom processors, and/or any suitable type of processor.

The processors 1002A-1002N and the main memory 1003 (or some sub-combination) can form a processing unit 1010. In some embodiments, the processing unit includes one or more processors communicatively coupled to one or more of a RAM, ROM, and machine-readable storage medium; the one or more processors of the processing unit receive instructions stored by the one or more of a RAM, ROM, and machine-readable storage medium via a bus; and the one or more processors execute the received instructions. In some embodiments, the processing unit is an ASIC (Application-Specific Integrated Circuit). In some embodiments, the processing unit is a SoC (System-on-Chip). In some embodiments, the processing unit includes one or more of the elements of the system.

A network device 1008 may provide one or more wired or wireless interfaces for exchanging data and commands between the system and/or other devices, such as devices of external systems. Such wired and wireless interfaces include, for example, a universal serial bus (USB) interface, Bluetooth interface, Wi-Fi interface, Ethernet interface, near field communication (NFC) interface, and the like.

Computer and/or Machine-readable executable instructions comprising of configuration for software programs (such as an operating system, application programs, and device drivers) can be stored in the memory 1003 from the processor-readable storage medium 1005, the ROM 1004 or any other data storage system.

When executed by one or more computer processors, the respective machine-executable instructions may be accessed by at least one of processors 1002A-1002N (of a processing unit 1010) via the communication channel 1001, and then executed by at least one of processors 1001A-1001N. Data, databases, data records or other stored forms data created or used by the software programs can also be stored in the memory 1003, and such data is accessed by at least one of processors 1002A-1002N during execution of the machine-executable instructions of the software programs.

The processor-readable storage medium 1005 is one of (or a combination of two or more of) a hard drive, a flash drive, a DVD, a CD, an optical disk, a floppy disk, a flash storage, a solid-state drive, a ROM, an EEPROM, an electronic circuit, a semiconductor memory device, and the like. The processor-readable storage medium 1005 can include an operating system, software programs, device drivers, and/or other suitable sub-systems or software.

As used herein, first, second, third, etc. are used to characterize and distinguish various elements, components, regions, layers and/or sections. These elements, components, regions, layers and/or sections should not be limited by these terms. Use of numerical terms may be used to distinguish one element, component, region, layer and/or section from another element, component, region, layer and/or section. Use of such numerical terms does not imply a sequence or order unless clearly indicated by the context. Such numerical references may be used interchangeable without departing from the teaching of the embodiments and variations herein.

As a person skilled in the art will recognize from the previous detailed description and from the figures and claims, modifications and changes can be made to the embodiments of the invention without departing from the scope of this invention as defined in the following claims.

We claim:

1. A system for an actuating needle manipulator with multiple degrees of freedom comprising:
a translational mechanism;
a remote center of motion (RCM) mechanism coupled to a distal end of the translational mechanism;
a needle insertion mechanism coupled to the distal end of the RCM mechanism;
a set of actuation couplers that are coupled to the translational mechanism and the RCM mechanism for integration with at least four degrees of freedom; and
an insertion actuation coupler coupled to the needle insertion mechanism.

2. The system of claim 1, wherein the translational mechanism, the RCM mechanism, and the needle insertion mechanism include degrees of freedom that are independently lockable.

3. The system of claim 1, wherein the translational mechanism comprises two multi-bar linkages arranged in a parallel configuration.

4. The system of claim 3, wherein the two multi-bar linkages comprise a first multi-bar linkage of the two multi-bar linkages is a five bar linkage and a second multi-bar linkage of the two multi-bar linkages is a six bar linkage with a parallelogram constraint.

5. The system of claim 4, further comprising an actuation cable system comprising a cable routed through the RCM mechanism and coupled to the needle insertion mechanism.

6. The system of claim 5, wherein each actuation coupler of the set of actuation couplers are coupled to a bar of the two multi-bar linkages, and the insertion actuation coupler is coupled to the needle insertion mechanism through the actuation cable system.

7. The system of claim 6, wherein the set of actuation couplers are grounded at or before a proximal end of the translational mechanism.

8. The system of claim 7, wherein the insertion actuation coupler is grounded at or before a proximal end of the translational mechanism.

9. The system of claim 7, wherein the insertion actuation coupler is positioned at a distal end of the translational mechanism.

10. The system of claim 9, further comprising a connecting member connecting the insertion actuation coupler to a proximal end of the translational mechanism.

11. The system of claim 5, wherein the cable is routed around revolute joints of the RCM mechanism.

12. The system of claim 11, wherein the cable is routed through revolute joints of the RCM in a configuration for constant cable length in motion of the RCM over a cable routing path.

13. The system of claim 11, wherein the revolute joints of the RCM mechanism are configured with cooperatively compensating radii.

14. The system of claim 11, wherein the revolute joints comprise of two revolute joints with a first radius and a second revolute joint with a second radius that is twice the first radius.

15. The system of claim 5, further comprising a set of hydraulic transmission interfaces that are each coupled to distinct bars of the two of multi-bar linkages, and an insertion actuation coupler that is a hydraulic transmission interface coupled to the needle insertion mechanism through the actuation cable system.

16. The system of claim 15, wherein the hydraulic transmission interfaces are active transmission interfaces.

17. The system of claim 1, wherein the set of actuation couplers and the insertion actuation coupler are hydraulic-to-mechanical transmission interfaces that convert between mechanical actuation and hydraulic actuation.

18. The system of claim 1, further comprising a set of hydraulic lines that connect to the set of actuation couplers and to the insertion actuation coupler.

19. The system of claim 1, further comprising motorized actuators connected to the actuation couplers.

20. The system of claim 1, further comprising sensors configured to monitor actuation through the actuation couplers.

21. The system of claim 1, wherein the translational mechanism actuates the four degrees of freedom with two translational actuation and two angular degrees of freedom; and wherein the needle insertion mechanism comprises one insertion degree of freedom.

22. The system of claim 1, wherein the needle insertion mechanism comprises an angled mounting structure establishing an angular offset from the distal end of the RCM mechanism, and which aligns a center point of rotation for an axis of insertion at a point below the system.

23. The system of claim 1, wherein the system is made of material consisting exclusively of magnetic resonance imaging (MRI) device compatible material.

24. A system comprising:
an input manipulator system and an output manipulator system, wherein each of the input manipulator system and the output manipulator system comprises:
a translational mechanism,
a remote center of motion (RCM) mechanism coupled to a distal end of the translational mechanism,
a needle insertion mechanism coupled to the distal end of the RCM mechanism, and
a set of actuation couplers that are coupled to the translational mechanism and the RCM mechanism for integration with at least four degrees of freedom, and
an insertion actuation coupler coupled to the needle insertion mechanism; and
a hydraulic transmission system, the hydraulic transmission system comprising: at least one hydraulic line and an intermediary active transmission interface serially integrated with the at least one hydraulic line, and wherein the hydraulic transmission system couples actuation of the first and manipulator system and the output manipulator system 120 through the at least one hydraulic line.
